(19) United States
(12) Patent Application Publication
(10) Pub. No.: US 2020/0121605 A1
(43) Pub. Date: Apr. 23, 2020

(54) METHODS AND COMPOSITIONS FOR LOCALIZED INTRADUCTAL DRUG DELIVERY TO THE BREAST AND REGIONAL LYMPH NODES

(71) Applicant: South Dakota Board of Regents, Pierre, SD (US)

(72) Inventors: Omathanu Perumal, Brookings, SD (US); Mibin Kuruvilla Joseph, Brookings, SD (US); Joshua Reineke, White, SD (US)

(21) Appl. No.: 16/659,297

(22) Filed: Oct. 21, 2019

Related U.S. Application Data
(60) Provisional application No. 62/747,977, filed on Oct. 19, 2018.

(51) Int. Cl. A61K 9/16; A61K 9/51; A61K 31/138; A61K 31/045; A61P 35/00; A61K 9/00; A61K 9/06

(52) U.S. Cl. CPC A61K 9/1647; A61K 9/5153; A61K 31/138; A61K 9/06; A61P 35/00; A61K 9/0019; A61K 31/045

(57) ABSTRACT

Disclosed herein is anticancer composition comprising an anti-cancer agent and poly(lactic-co-glycolic acid) (PLGA) carrier thereof. Further disclosed herein is a method for treating a breast disorder in a subject in need thereof, comprising administering to the breast of a subject via an intraductal injection an effective amount of a composition comprising a therapeutic agent and a PLGA carrier thereof. In some aspects the carrier is a microsphere.

### CROSS-REFERENCE TO RELATED APPLICATION(S)

This application claims priority to U.S. Provisional Application No. 62/747,977, filed Oct. 19, 2018, and entitled “Methods and Compositions for Localized Intraductal Drug Delivery to the Breast and Regional Lymph Nodes,” which is hereby incorporated by reference in its entirety under 35 U.S.C. § 119(e).

### FIELD OF THE INVENTION

Disclosed herein are methods and compositions for treating breast cancer and other breast disorders.

### BACKGROUND OF THE INVENTION

Breast cancer is the second most commonly diagnosed cancer among women. More than 95% of breast cancers originate from the epithelial cells lining the milk ducts. Current therapeutic approaches include systemic chemotherapy, radiation, hormonal therapy and surgical procedures (Breast conservation surgery, Mastectomy) all of which are associated with significant side effects. Direct intraductal injection into the breast has the potential to localize drug to the breast and minimize systemic side-effects. However, the limited retention of free drug in the ducts and the frequent injection required to sustain drug levels are major challenges in translating this approach for clinical application. Accordingly, there is a need in the art for improved drug compositions and delivery methods to improve drug retention in the ducts to enhance targeted drug efficacy and minimize systemic side effects.

### SUMMARY OF THE INVENTION

Disclosed herein is anticancer composition comprising an anti-cancer agent and poly(lactic-co-glycolic acid) (PLGA) carrier thereof. In certain aspects, the carrier is a microsphere. In further aspects, the microsphere is comprised of a polymer of about 75-85 KDa. In yet further aspects, the particle size of the microsphere ranges from about 1 to about 50 μm. In yet further aspects, the PLGA is comprised of lactic acid and glycolic acid present at a ratio of about 75:25.

According to certain further aspects, the carrier is a nanoparticle with a size ranges from about 1 to about 1000 nm. In exemplary implementations, the particle size of the nanoparticle is approximately 200 nm.

In further aspects, the composition further comprises a thermogel. In exemplary implementations, the thermogel is comprised of poly(ε-caprolactone-co-lactide)-b-poly(ethylene glycol)-b-poly(ε-caprolactone-colactide) (PCLA-PEG-PCLA). In certain aspects, the PLGA carrier is dispersed within the thermogel. In exemplary implementations of these embodiments, the PLGA carrier is in the form of microspheres, nanoparticles, or combinations thereof. In certain aspects, the PLGA is present at about 10% w/w of the thermogel composition. According further exemplary implementations, the thermogel is comprised of a polymer with a PCLA:PEG:PCLA molecular weight ratio of 1700:1500:1700 Da, and the thermogel exhibits sustained release of the anti-cancer agent upon injection into a subject. In certain aspects, the anti-cancer agent is tamoxifen and the delivery of the composition to the subject produces sustained exposure of the site of delivery to 4-hydroxy tamoxifen and endoxifen.

Further disclosed herein is a method for treating a breast disorder in a subject in need thereof comprising the steps of administering to the breast of a subject, via an intraductal injection, an effective amount of a composition comprising a therapeutic agent and a PLGA carrier thereof. In certain aspects, the composition forms an in situ gel implant upon injection into the subject and the composition is retained in the breast duct and exhibits sustained release of the therapeutic agent therein. According to certain aspects, the breast disorder is breast cancer and the therapeutic agent is an anti-cancer agent. In exemplary implementations of these embodiments, the anti-cancer agent is select from a list consisting of selective estrogen receptor modulators (e.g. tamoxifen, 4-hydroxy tamoxifen, endoxifen, fulvestrant), retinoids (e.g. fenretinide), chemotherapeutic agents (e.g. 5-fluorouracil, paclitaxel, cyclophosphamide) and Herceptin. In certain aspects, the anti-cancer agent is a combination of two or more of the foregoing agents.

According to certain alternative embodiments, the breast disorder is an infection. In exemplary implementations of these embodiments, the therapeutic agent is an antibiotic or an anti-inflammatory agent. According to still further aspects, the disclosed method further comprises the step of administering the composition in conjunction with at least one other treatment or therapy. In exemplary aspects, the step of administering another treatment or therapy comprises co-administering an anti-cancer agent. In further aspects, the other treatment or therapy comprises co-administering α-santalol.

Further disclosed herein is a method for treating a lymph node disorder in a subject in need thereof comprising administering to the breast of a subject via an intraductal injection an effective amount of a composition comprising a therapeutic agent and a PLGA carrier thereof and wherein the composition is retained in the lymph node and exhibits sustained release of the therapeutic agent therein. In exemplary aspects, the lymph node disorder is selected from a list consisting of: lymphedema, lymphadenopathy, lymphadenitis, lymphomas, and lymphoproliferative disorders.

### BRIEF DESCRIPTION OF THE FIGURES

FIG. 1 shows representative images of Intraductal retention of polystyrene particles using Bruker whole body imaging system from 0-72 hours. (n=3).

FIG. 2 shows the fluorescence Intensity profile of polystyrene carrier systems plotted as percentage of maximum Intensity. The graph depicts the influence of particle size on intraductal retention of polystyrene nanoparticles. Data=Mean±SEM, n=3.

FIG. 3 the physicochemical characteristics of long acting PLGA formulations. Data=Mean±SD, n=3.

FIG. 4 shows representative Scanning Electron Microscopy (SEM) images of PLGA formulations (Microspheres and PLGA In situ forming implant and Nanoparticles).

FIG. 5 shows In Vitro release profiles of microspheres (PLGA and PDLLA), nanoparticles and In situ forming implants (0-96 hours). Data=Mean±SD, n=3.

FIG. 6 shows representative fluorescence (Cy 5.5 dye) images showing intraductal retention of different PLGA formulations captured using Bruker In Vivo Xtreme II whole body imaging system.

FIG. 7 shows the fluorescence intensity profile of Cy 5.5. loaded PLGA formulations expressed as percentage of maximum fluorescence intensity. Data point=Mean±SEM, n=3.

FIG. 8 shows photographs confirming intraductal localization of PLGA formulations using crystal violet.

FIG. 9 shows mammary whole mounts showing the localization of PLGA formulations in the breast ducts. The panel on the top represents phase contrast images and the corresponding fluorescence images are shown at the bottom. The arrow in the inset indicate the particles retained within the breast ducts.

FIG. 10 shows fluorescence images of the excised mammary glands at 96 hrs (top panel) and the corresponding brightfield and fluorescence images of PLGA and PDLLA formulations. The images were captured using confocal microscopy under 20× objective.

FIG. 11 shows an image of the axillary lymph node in female Sprague Dawley rats.

FIG. 12 shows fluorescence images of mammary gland and axillary lymph node localization of PLGA microspheres and nanoparticles from 1-48 hours. Biodistribution of formulations after intraductal injection is also depicted. Images were captured using Bruker In Vivo Xtreme II (n=3). Panel ‘a’ shows the lymphoid organs with the excised mammary gland and Panel ‘B’ shows the biodistribution in other organs (1—Liver, 2 Spleen, 3—Lymph node (LN), 4—Mammary gland (MG), 5—Kidneys. 6-Heart, 7-Lungs).

FIG. 13 shows fluorescence images of mammary gland and axillary lymph node localization of PLGA in-situ implant and free dye from 1-48 hours. Biodistribution of formulations after intraductal injection is also depicted. Images were captured using Bruker In Vivo Xtreme II (n=3). Panel ‘a’ shows the lymphoid organs with the excised mammary gland and Panel ‘B’ shows the biodistribution in other organs (1—Liver, 2 Spleen, 3—Lymph node (LN), 4—Mammary gland (MG), 5—Kidneys. 6—Heart, 7—Lungs).

FIG. 14 shows representative images of histology sections of mammary glands after 7 days of treatment (PLGA formulations) and viewed under 20× objective.

FIG. 15 shows images showing intraductal retention of PLGA formulations in porcine breast after 4 days.

FIG. 16 shows TMX release from PLGA microspheres (homogenization vs overhead stirring).

FIG. 17 shows TMX release PLGA (75-85 KDa) microspheres (homogenization vs Overhead stirring).

FIG. 18 shows TMX release from PDLLA (55-65 KDa) microspheres (homogenization vs overhead stirring).

FIG. 19 shows TMX release from PLGA/PDLLA microspheres.

FIG. 20 shows the effect of drug polymer ration on TMX release from PLGA nanoparticles.

FIG. 21 shows tamoxifen release profile form in situ forming.

FIG. 22 shows the release of tamoxifen from formulations of different particle sizes formed using homogenization and overhead stirring. Each Value is Mean±SD.

FIG. 23 shows the in vitro release profile of tamoxifen from optimized PLGA nanoparticles of particle size 274.1±4.87. Each Value is Mean±SD.

FIG. 24 shows the In Vitro release profile of tamoxifen from PLGA in situ gel. PLGA (LA:GA 50:50) (M_{w} 5-10 KDa) and PLGA (LA:GA 75:25) (M_{w} 10-15 KDa). Each Value is Mean±SD.

FIG. 25 shows Scanning Electron Microscope images of optimized formulations of PLGA in-situ gel, microspheres and nanoparticles.

FIG. 26 shows the plasma profile of tamoxifen after intraductal injection of formulations.

FIG. 27 shows the profile of 4-hydroxytamoxifen after intraductal injection of PLGA formulations.

FIG. 28 shows the plasma profile of Endoxifen after intraductal injection of PLGA formulations.

FIG. 29 shows the breast concentration of tamoxifen in the mammary glands at different time points (12, 24, 48, 72, 144, 168, 240 and 336 hours). ISG is in-situ gel. Each value is Mean±SD, n=3.

FIG. 30 shows the breast concentration of 4-hydroxytamoxifen at different time points (12, 24, 48, 72, 144, 168, 240 and 336 hours). Each value is Mean±SD, n=3.

FIG. 31 shows lymph node concentration of Tamoxifen at different time points (12-336 hrs). Each Value is Mean±SD, n=3.

FIG. 32 shows the lymph node concentration of Endoxifen at different time points (12-336 hrs). Each Value is Mean±SD, n=3.

FIG. 33 shows the lymph node concentration of 4-Hydroxytamoxifen at different time points (12-336 hrs). Each Value is Mean±SD, n=3.

FIG. 34 shows the biodistribution of intraductal free tamoxifen at the end of the treatment. Each value is Mean±SD, n=3.

FIG. 35 shows the biodistribution of Intraductal PLGA Nanoparticles at the end of the treatment. Each Value is Mean±SD, n=3.

FIG. 36 shows the biodistribution of Intraductal PLGA Microspheres at the end of the treatment. Each Value is Mean±SD, n=3.

FIG. 37 shows the biodistribution of Intraductal PLGA in-situ gel at the end of the treatment. Each Value is Mean±SD, n=3.

FIG. 38 shows a scanning electron microscopy image of 4-hydroxy tamoxifen loaded PLGA microspheres.

FIG. 39 shows PLGA microspheres dispersed in PCLA-PEG-PCLA Thermogel before and after incubation at 37° C.

FIG. 40 shows an in vitro release profile of 4-hydroxy tamoxifen from PLGA microspheres, PCLA-PEG-PCLA Thermogel and PLGA Microspheres in PCLA-PEG-PCLA Thermogel formulations. PCLA-PEG-PCLA is poly(ε-caprolactone-co-lactide)-b-poly(ethylene glycol)-b-poly(ε-caprolactone-co-lactide).

FIG. 41 shows the rat mammary gland (MG) concentration of 4-hydroxytamoxifen treated with PLGA microspheres in PCLA-PEG-PCLA thermogel after 7, 14 and 28 days. Control MG is the contralateral untreated mammary gland.

FIG. 42 shows the rat mammary gland concentration of endoxifen (metabolite generated from 4-hydroxy tamoxifen) treated with PLGA microspheres in PCLA-PEG-PCLA thermogel after 7, 14 and 28 days. Control MG is the contralateral untreated mammary gland.

FIG. 43 shows the rat mammary gland concentration of 4-hydroxy tamoxifen treated with free 4-hydroxytamoxifen after 7, 14 and 28 days. Control MG is the contralateral untreated mammary gland.

FIG. 44 shows the rat plasma concentration of 4-hydroxytamoxifen treated with PLGA microspheres in PCLA-PEG-PCLA thermogel after 7, 14 and 28 days. Control is untreated rat plasma.

FIG. 45 shows the rat plasma concentration of endoxifen (metabolite generated from 4-hydroxy tamoxifen) treated with PLGA microspheres in PCLA-PEG-PCLA thermogel after 7, 14 and 28 days. Control is untreated rat plasma.

FIG. 46 shows the rat plasma concentration of 4-hydroxytamoxifen treated with free 4-hydroxytamoxifen after 7, 14 and 28 days. Control is untreated rat plasma.

FIG. 47 shows the plasma concentration of endoxifen (metabolite generated from 4-hydroxy tamoxifen) treated with free 4-hydroxytamoxifen after 7, 14 and 28 days. Control is untreated rat plasma.

FIG. 48 shows the lymph node concentration of 4-hydroxytamoxifen in rats treated with PLGA Microspheres in PCLA-PEG-PCLA Thermogel at Days 7 and 14.

FIG. 49 shows the lymph node concentration of endoxifen (metabolite generated from 4-hydroxy tamoxifen) in rats treated with PLGA Microspheres in PCLA-PEG-PCLA Thermogel at Days 7 and 14.

FIG. 50 shows the organ distribution of 4-hydroxytamoxifen (4-HT) and endoxifen (EDX-metabolite generated from 4-hydroxy tamoxifen) in rats treated with free 4-hydroxy tamoxifen and PLGA microspheres in PCLA-PEG-PCLA thermogel at Days 7 and 14.

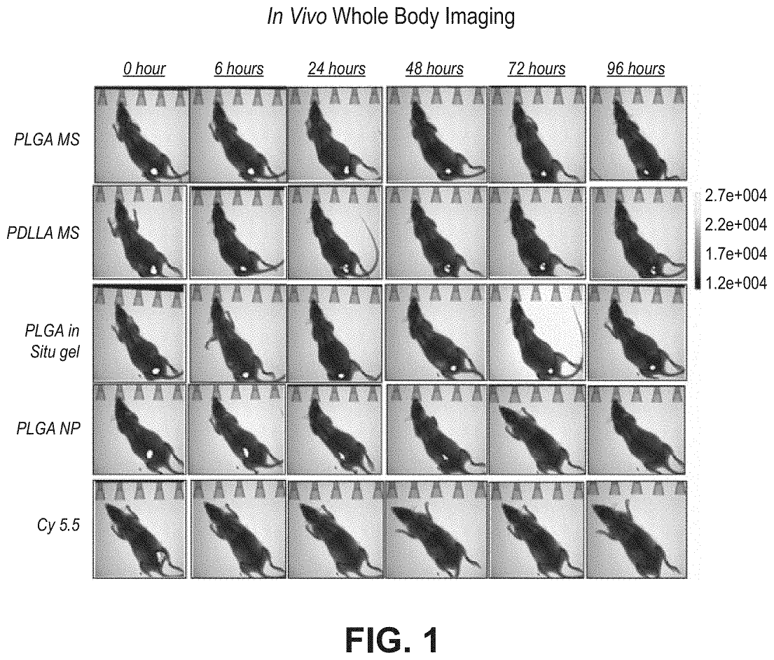

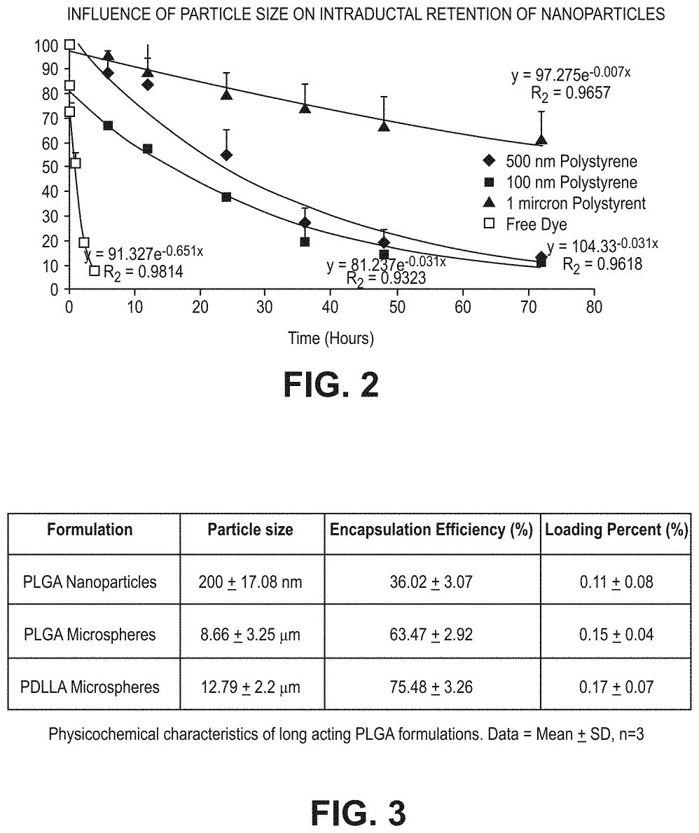

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The term “substantially” is defined as being largely but not necessarily wholly what is specified (and include wholly what is specified) as understood by one of ordinary skill in the art. In any disclosed embodiment, the term “substantially” may be substituted with “within [a percentage] of” what is specified, where the percentage includes 0.1, 1, 5, and 10 percent.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more—OCH2CH2O— units in the polyester, regardless of whether ethylene glycol was used to prepare the polyester. Similarly, a sebacic acid residue in a polyester refers to one or more—CO(CH2)8CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

As used herein, the term “breast disorders” include breast cancers and benign but often precancerous lesions, such as ductal hyperplasia, lobular hyperplasia, atypical ductal hyperplasia, and atypical lobular hyperplasia. Breast cancers include any malignant tumor of breast cells. There are several types of breast cancer. Exemplary breast cancers include, but are not limited to, ductal carcinoma in situ, lobular carcinoma in situ, invasive (or infiltrating) ductal carcinoma, invasive (or infiltrating) lobular carcinoma, inflammatory breast cancer, triple-negative breast cancer, ER+ breast cancer, HER2+ breast cancer, adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, and micropapillary carcinoma. A single breast tumor can be a combination of these types or be a mixture of invasive and in situ cancer. Breast disorders also include conditions such as cyclic mastalgia (mastitis) in women, gynecomastia in men and mastitis in animals.

As used herein, the term “subject” refers to the target of administration, e.g., an animal. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more breast disorders prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to a physical examination by a person of skill, for example, a physician, and found to have a condition that can be diagnosed or treated by a compound or composition that can reduce tumor size or slow rate of tumor growth. A subject having cancer, tumor, or at least one cancer or tumor cell, may be identified using methods known in the art. For example, the anatomical position, gross size, and/or cellular composition of cancer cells or a tumor may be determined using contrast-enhanced MRI or CT. Additional methods for identifying cancer cells can include, but are not limited to, ultrasound, bone scan, surgical biopsy, and biological markers (e.g., serum protein levels and gene expression profiles). An imaging solution comprising a cell-sensitizing composition of the present invention may be used in combination with MRI or CT, for example, to identify cancer cells.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. In preferred embodiments, the disclosed compositions are administered to the breast of a subject through intraductal injection. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

The phrase “anti-cancer composition” can include compositions that exert antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agents available in commercial use, in clinical evaluation and in pre-clinical development, which could be included in this application by combination drug chemotherapy. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

The compound “santalol” refers to both alpha-santalol and beta santalol. α-Santalol is a natural terpene. The liquid α-santalol is the major constituent (≈61%) of the essential oil of Sandalwood oil. While both enantiomers can be effective for treating various conditions, alpha-santalol has been found to be suitably effective as described herein. The chemical structure of alpha-santalol is:

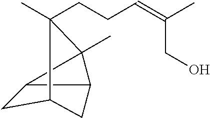 [SMILES: C/C(=C/CCC1(C)C2CC3C(C2)C31C)CO]

The chemopreventive properties of α-santalol against both chemical and UV-induced skin cancer have been extensively studied (Dwivedi, C, Abu-Ghazaleh, A.; Eur. J. Cancer Prev. 1997; 6:399-401). α-santalol has also been demonstrated to have efficacy in the treatment of breast cancer. See, e.g. U.S. Pat. No. 9,220,680, which is incorporated herein by reference for all purposes.

The “mammary papilla” or “nipple” is a projection on the breast used for delivering milk to offspring by female mammals. Milk is produced in lobules of the mammary glands and the milk is delivered via ducts that open on the surface of the mammary papilla. The mammary papilla is mainly composed of the epidermis and the dermis. Each mammary papilla includes approximately 10-15 ducts that lead from the surface of the mammary papilla to various lobules. Mammalian ducts are typically about 10-60 microns in diameter. Corneocytes, which are stratified keratinocytes, are the cells mainly responsible for the barrier function of the skin. The corneocytes of the mammary papilla epidermis are smaller and less concentrated than the corneocytes for other skin (600 corneocytes per cm² for mammary papilla and 800 corneocytes per cm² for normal skin). This difference results in the mammary papilla being a more permeable tissue than normal skin. There are also fewer layers of corneocytes in the mammary papilla compared to normal skin, resulting in a higher rate of transepidermal water loss compared to normal skin, indicating the less obstructive nature of the mammary papilla.

The majority of breast cancers originate in the epithelial cells lining the ducts in the breast. Therefore, localized delivery of chemopreventive/chemotherapeutic agents could be a promising approach for prevention and treatment of breast cancer (Lee et al., Int. J. Pharm. 2010, 387, (1-2), 161-166). The mammary papilla is the exit point for delivering milk through ducts produced in globules. The openings on the surface of the mammary papilla are in the size range of 50-60 μm (Rusby et al., Breast Cancer Res. Treat. 2007, 106, (2), 171-179).

Furthermore, the epidermis is thinner in the mammary papilla compared to the surrounding breast skin (14 layers of corneocytes compared to 17) (Kikuchi et al., Br. J. Dermatol. 2011, 164, (1), 97-102). Therefore, by topical application on the mammary papilla, therapeutic agents can be directly delivered to the ducts and lobules in the breast. This invention provides compositions and methods of using mammary papilla as a route for localized drug delivery to the breast. In certain embodiments, this delivery is achieved through the use of microneedles placed on the nipple. Such techniques are described in U.S. Pat. No. 9,220,680, which is incorporated herein by reference for all purposes.

According to certain embodiments of the disclosed compositions and methods, compositions are administered to the subject by way of intraductal injection through the nipple by use of catheter or needle. Such approaches are described in Stearns V et al., Preclinical and Clinical Evaluation of Intraductally Administered Agents in Early Breast Cancer. SCI TRANSL MED. (2011) October 26; 3(106), and Murata, S. et. al., Ductal Access For Prevention and Therapy of Mammary Tumors, CANCER RES. (2006) Jan. 15; 66(2):638-45, each of which is incorporated by reference herein in its entirety.

Disclosed herein is an anticancer composition comprising an anti-cancer agent and a poly(lactic-co-glycolic acid) (PLGA) carrier thereof. In certain aspects, the composition of PLGA can vary with the ratio of lactic acid:glycolic acid from 50:50, 60:40, 75:25, 85:15 or 100% poly lactic acid or poly glycolic acid. In certain aspects, the PLGA is comprised of lactic acid and glycolic acid, present at a ratio of about 75:25.

The PLGA ratio influences crystallinity, solubility, rate of degradation and drug release. For example, the higher the lactide content, slower is the degradation vis-à-vis drug release. Poly lactic acid contains an asymmetric α-carbon which is typically described as the D or L form in classical stereochemical terms and sometimes as R and S form, respectively. The enantiomeric forms of the polymer PLA are poly D-lactic acid (PDLA) and poly L-lactic acid (PLLA). PLGA is generally an acronym for poly D,L-lactic-co-glycolic acid where D- and L-lactic acid forms are in equal ratio. The physicochemical properties of optically active PDLA and PLLA are nearly the same. In general, the polymer PLA can be made in highly crystalline form (PLLA) or completely amorphous (PDLA) due to disordered polymer chains. PGA is void of any methyl side groups and shows highly crystalline structure in contrast to PLA.

In certain aspects, the carrier is a microsphere. The molecular weight of the polymer can vary from about 5 KDa up to about 160 KDa. The molecular weight influences the viscosity, degradation profile, particle size and drug release. For example, an increase in molecular weight will increase viscosity and decrease polymer degradation/drug release. According to certain aspects, PLGA microspheres have a molecular weight from about 75 KDa to about 85 KDa. In certain aspects, the particle size of the microsphere ranges from about 0.05 to about 200 μm. According to further aspects, the particle size of the microsphere ranges from about of 0.05 to 50 μm. In yet further aspects, the particle size of the microsphere is approximately 9 μm. According to further aspects, the carrier is a nanoparticle. In exemplary embodiments of these aspects, the particle size of the nanoparticle ranges from about 1 to about 1000 nm. In yet further aspects, the particle size of the nanoparticle ranges from about 50 to about 999 nm. In still further aspects, the particle size of the nanoparticle is approximately 200 nm. In yet further aspects, the composition forms an in situ gel implant upon administration to the subject.

According to still further embodiments, the composition further comprises a gel (e.g., a thermogel). In exemplary implementations of these embodiments, the disclosed PLGA microparticles and/or nanoparticles are dispersed within the gel to allow for target and/or sustained delivery of the anti-cancer composition to site of action. In certain aspects, the gel is comprised of poly(ε-caprolactone-co-lactide)-b-poly(ethylene glycol)-b-poly(ε-caprolactone-colactide) (referred to herein as “PCLA-PEG-PCLA”). In exemplary implementations, the PCLA-PEG-PCLA are present at a molecular weight ratio of 1700:1500:1700 Da. The molecular weight and the gel concentration are critical for forming a thermogel (Sol-gel transition temperature) at the body temperature. Similarly the molecular weight and the gel percentage have an influence on gel viscosity and drug release. The higher the molecular weight, slower is the drug release and higher the viscosity. Generally increasing the molecular weight lowers the sol-gel transition. In exemplary embodiments, the polymer has a sol-gel transition around 30° C.

According to certain alternative embodiments, the thermagel may be comprised of polymers including, but not limited to: PLGA-PEG-PLGA, PDLLA-PEG-PDLLA, PCL-PEG-PCL, PCLA-PEG-PCLA, PLA-PEG-PLA. According to these implemenations, the molecular weight of PLGA ranges from about 500-5000 Da; the molecular weight of PEG ranges from about 400-5000 Da and the molecular weight of PCL ranges from about 500-5000 PCL.

In further implementations, the w/w % ratio of L:G (Lactide:Glcyolide) range: Lactide—about 50-90% w/w and Glycolide about 50-10% w/w. In further implementations, LA:CL (Lactide:Caprolactone) range—about 50-50%, 75:25, 90:10 w/w.

The polymer concentration range used for gel formation is 10% w/v to 40% w/v. From an intraductal injectability standpoint 20-25% w/v gel concentration is optimal. In further implementations, PCLA-PEG-PCLA is prepared at about 25% w/v in an aqueous solvent. In certain implementations, the aqueous solvent is DI water.

According to certain implementations, the disclosed gels can formed by different types of polymers using multiple approaches. According to certain embodiments, PLGA is dissolved in organic solvents such as N-methyl pyrrolidone, followed by phase separation when diluted with aqueous medium at the injection site in the body (in-situ gel based on phase separation). According to further embodiments, the gel is an aqueous based thermogel (e.g. gel is formed due to the difference in room and body temperature) was formed using block copolymer as described further below in the 4-hydroxy tamoxifen example. According to still further embodiments, hydrogels, organogels (gels with organic solvent) using other natural or synthetic polymers can be used.

According to certain embodiments, PLGA microspheres are loaded into the PCLA-PEG-PCLA at about 10% w/w. In further embodiments, the PLGA microspheres are loaded into the gel at from about 1% to about 25% w/w.

According to certain embodiments, microspheres are loaded into the disclosed gels. According to further embodiments, nanospheres are loaded in to the disclosed gels. In still further embodiments, a combination of nanospheres, microspheres and/or free therapeutic composition are loaded into the disclosed gels. As will be appreciated, the nanoparticles and microspheres offers the advantages of avoiding drug solubility and drug loading issues in the gel. More importantly, the microspheres or nanoparticles offer the advantages of controlling the drug release for much prolonged periods. i.e. drug release is in the following decreasing rank order: free drug in the gel>Nanoparticles in the gel>Microspheres in the gel.

Also disclosed herein is a method for treating a breast disorder in a subject in need thereof comprising administering to the breast of a subject via an intraductal injection an effective amount of a composition comprising a therapeutic agent and a PLGA carrier thereof. In certain aspects, the carrier is a microsphere. In further aspects, the carrier is a nanoparticle. In yet further aspects, the composition forms an in situ gel implant upon injection into the subject.

According to certain embodiments, the composition is administered in a prophylactically effective amount. According to exemplary aspects of these embodiments, the composition is administered to subjects at high risk of developing a breast disorder.

In certain aspects, the breast disorder is breast cancer and the therapeutic agent is an anti-cancer agent.

In further aspects, the breast disorder is an infection and the therapeutic agent is an antibiotic or anti-inflammatory agent.

According to certain alternative embodiments, the disclosed method is useful for the targeting drugs to, and effectuating sustained release in, the regional lymph nodes. According to these embodiments, compositions administered through intraductal injection drain into the region lymph node where the composition is retained and effectuates sustained release of the therapeutic agent in the lymph nodes. These embodiments show particular utility for the treatment of conditions including, but not limited to, lymphadenitis.

According to certain embodiments, the method further comprises administering the composition in conjunction with at least one other treatment or therapy. In certain aspects, the other treatment or therapy comprises co-administering an anti-cancer agent. In exemplary embodiments, the other treatment or therapy comprises co-administering α-santalol. In certain aspects, the composition is administered in a therapeutically effective amount. In further aspects, the composition is administered in a prophylactically effective amount.

In further aspects, the composition administered to the subject may be in a range of about 0.001 mg/kg to about 1000 mg/kg.

According to certain embodiments, the disclosed method further comprises administering the composition in conjunction with at least one other treatment or therapy. In certain aspects, the at least one other treatment or therapy comprises co-administering an anti-neoplastic agent. In certain aspects, the other treatment or therapy is chemotherapy.

According to certain further embodiments, the method further comprises diagnosing the subject with cancer. In further aspects, the subject is diagnosed with cancer prior to administration of the composition. According to still further aspects, the method further comprises evaluating the efficacy of the composition. In yet further aspects, evaluating the efficacy of the composition comprises measuring tumor size prior to administering the composition and measuring tumor size after administering the composition. In even further aspects, evaluating the efficacy of the composition occurs at regular intervals. According to certain aspects, the disclosed method further comprises optionally adjusting at least one aspect of method. In yet further aspects, adjusting at least one aspect of method comprises changing the dose of the composition, the frequency of administration of the composition, or the route of administration of the composition.

The disclosed compositions and methods provide are characterized by at least the following:
1. Critical particle size and gel formulation for retention in the breast and lymph node
2. Formulation composition for prolonged drug release in the breast and lymph nodes
3. In certain embodiments, where tamoxifen is employed as the anti-cancer agent, the advantage of delivering tamoxifen is that generates two active metabolites (4-hydroxy tamoxifen and endoxifen) in the breast and/or lymph nodes. Since metabolite generation is a time dependent process, it is critical to retain and prolong the drug release in the breast and lymph nodes to generate the active metabolites. Sufficient metabolite levels cannot be achieved when the free tamoxifen is injected. The generation of active metabolites from tamoxifen formulations is expected to produce a significantly higher efficacy compared to just free tamoxifen. There is hardly any information in the literature with regard to the generation of active metabolites in the breast and more specifically in the lymph nodes using formulation approaches. In essence you get active moieties with the injection of one formulation. The same is true for 4-hydroxy tamoxifen formulations with regard to generation of endoxifen, e.g. two active moieties are achieved with the injection of a single formulation.
4. In certain embodiments, the disclosed the formulation composition (particle size and formulation viscosity) is critical for localized delivery to the breast/lymph nodes as wells as for generation of sufficient levels of active metabolites. Additionally, the disclosed formulation results in much lower systemic exposure resulting in reduced side effects.

Overall the formulation composition (particle size and formulation viscosity) is critical for localized delivery to the breast/lymph nodes as wells as for generation of sufficient levels of active metabolites. Additionally, the formulation results in much lower systemic exposure resulting in reduced side effects

Application of the disclosed compositions and methods may be implemented in one or more of the following embodiments:
Formulation and in-vivo evaluation of anti-cancer drugs including selective estrogen receptor modulators (e.g. tamoxifen, 4-hydroxy tamoxifen, endoxifen, fulvestrant), retinoids (e.g. fenretinide), chemotherapeutic agents (e.g 0.5-fluorouracil, paclitaxel, cyclophosphamide), Herceptin, among other anti-cancer agents;
Variation of molecular weight of the polymers, copolymer ratio, different methods of incorporation (e.g. nanoparticles, microspheres) and viscosity to further prolong the retention and drug release in the breast and lymph node;
Use of other biodegradable and non-degradable synthetic or natural polymers such as polycaprolactone, polyesters, polyanyhdrides, cellulose derivatives, chitosan, zein, albumin, gelatin, etc.;
Use of lipid matrices such as liposomes, solid lipid nanoparticles, emulsions, etc.;
Use of other injectable gels including HPMC gels or thermo reversible gels (e.g. polaxamer);
Use of other particulate systems including microspheres, liposomes, micelles, and nanoparticles;
Use of polymeric drug conjugates including linear or branched polymeric-drug conjugates, e.g. dendrimers, PEG, PLGA, etc.;
Use of lipophilic prodrugs or slowly dissolving prodrugs, salts or oil soluble salts;
Use of oily vehicles to sustain the drug release e.g. cotton seed oil
Use of polymeric drug conjugates encapsulated in particulate systems such as microspheres, nanoparticles, liposomes or micelles; and
Use of polymeric drug conjugates encapsulated in particulate systems and dispersed in an injectable gel formulation, which will further prolong the drug release from weeks to months.

### Examples

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

The following examples demonstrate that the disclosed compositions and methods are characterized by the following features:
Particle size of the formulation is critical for injectability and prolonged retention in the breast as well as the regional lymph nodes. We have identified that the particle size of the polymeric particles should be >500 nm for prolonged retention (>2 days) in the breast.
Gels are suitable formulation matrices to prolong the drug retention in the breast. The gel should have optimal viscosity for achieving sustained drug release and injectability.
The particulate systems drain to the regional lymph nodes to prevent the metastasis of breast cancer.
The drug levels can be sustained from several weeks to up to several months.
Formulation can be tailored for various applications by controlling the particle size, drug release, formulation compositions and viscosity to achieve prolonged retention and sustained drug levels in the breast and the regional lymph nodes.

In this study we used US-FDA approved biodegradable synthetic polymers. Polyesters such as Poly (D,L-lactic acid) (PLA) and poly lactic-co-glycolic acid (PLGA) which was used in our study have been used for developing depot preparations because of its biodegradability, tunable physicochemical properties and the ability to microspheres and nanoparticles (11, 12). We also used in-situ polymeric implants to retain and prolong the drug release. These gel systems once injected into the body, get in contact with physiological fluids to form a semi-solid implant with the drug entrapped in the formulation matrix.

### I. Intraductal Injection of Polystyrene Nanocarriers

Aim:

To determine the influence of particle size on the intraductal retention of model polystyrene particles (100 nm, 500 nm and 1 μm).

METHODOLOGY: Female Sprague Dawley rats (3-4 weeks old) were injected with polystyrene nanocarrier systems of different particle sizes (100 nm, 500 nm, 1 μm) intraductally under a surgical microscope. The animals were imaged at predetermined time points using Bruker In Vivo Extreme II whole body animal imaging system for 72 hours. The following instrument settings were used for the study. Mode: High speed, FOV 15, Excitation/Emission 730/790 nm and f stop 1.1. The images were processed using the Bruker Xtreme II imaging software. The fluorescence intensities were plotted as percentage of maximum fluorescence intensity for respective treatment groups using a fixed ROI (region of interest) for each of the treatment group to normalize the data and eliminate bias.

### Intraductal Retention of Polystyrene Nanocarriers Using Whole Body Imaging System

RESULTS: From the study, we found that particle size influenced the retention of polystyrene carrier systems in the ducts. Larger particles were found to be retained longer in the ducts. When plotted as percentage of maximum fluorescence intensity, 60% of larger 1 μm particles were retained in the ducts at the end of 72 hours. Smaller (100 nm and 500 nm) particles were found to diffuse out of the ducts after 48 hours with no fluorescence signals detected at 72 hours. The free dye was found to diffuse out of the ducts in 4 hours.

### II. Intraductal Injection of Cy5.5 Labelled PLGA Formulations

Aim:

To test the influence of formulation on intraductal retention using a US-FDA approved polymer and a model hydrophobic Near IR dye (Cy5.5)

Methodology

Formulation of Microspheres

Microspheres were prepared by oil-in-water (O/W) emulsion method (1) with slight modifications. Polymer (500 mg) and Cy5.5 (1 mg) were dissolved in (5 ml) methylene chloride (DCM). This organic phase was then added drop wise into an aqueous phase containing 1% PVA. The mixture was then homogenized at 10,000 rpm for 1 minute to form microspheres. The emulsion was then stirred at 300-400 rpm for 3 hours to remove DCM. As the solvent was being removed, the emulsifier maintained the spherical configuration of the oil droplets and the microspheres were hardened as discrete particles. The microspheres were then collected by centrifugation at 4000 rpm for 20 minutes. The particles were washed, freeze dried, lyophilized, and stored at 4° C. and the lyophilized preparation was then used for further studies.

Formulation of Nanoparticles

PLGA nanoparticles were prepared by emulsion solvent evaporation method with slight modifications. Briefly, 100 mg of PLGA was dissolved in 4 ml methylene chloride containing Cy5.5 (1 mg). The organic phase was then added drop wise into an aqueous phase containing 1% PVA. The mixture was then sonicated using a probe sonicator set at 50 W of energy output for 1 minute to form oil-in-water (o/w) emulsion. The emulsion was then stirred at 300-400 rpm for 2-3 hours to remove methylene chloride. The nanoparticles were separated by ultracentrifugation (20,000 rpm) for 20 minutes. The particles were collected, washed and freeze dried and the lyophilized powder was stored at 4° C. for further use.

Formulation of In Situ Forming Implants

PLGA in situ forming gels were prepared by simply dissolving the polymer into a highly water miscible solvent like N-methyl-2-pyrrolidone (NMP). Briefly, 15 wt % PLGA was used to formulate in situ forming gels. In this study, PLGA of two copolymer (LA:GA) ratios, 50:50 and 75:25 of molecular weights 5-10 KDa and 10-15 KDa respectively were used to formulate the implants with desired characteristics for intraductal injections. Cy5.5 in NMP was dispersed into the polymer phase and this was further used for intraductal injections. The blending of PLGA of different molecular weights was used to obtain the required release profiles.

In Vitro Release Study

In Vitro release study was conducted using a previously established method with slight modifications. Briefly, 5-10 mg of microspheres and nanoparticles were dispersed in 1 ml of release medium (PBS, pH 7.4 containing 1% w/v Tween 80) in an Eppendorf tube. At each time point, the tubes were centrifuged (10,000 rpm) and 1 ml of the supernatant was collected. The release medium was replaced with 1 ml fresh release medium to maintain sink conditions. The supernatant was then analyzed using UV spectroscopy to determine the dye content in the release medium. For In situ forming implants, 10 μg equivalent of gel was injected into 1 ml of release medium using 27-31 G needle to form implants and the release studies were carried out as described above.

Determination of particle size: The particle size of microspheres were determined using Smart Tiff V03, by randomly counting 50-70 particles in two to three SEM images. The particles size of nanoparticles were analyzed using DLS (dynamic light scattering) technique. For studying the morphology of in situ implants, the formulation was injected into the release medium, allowed to form the implant (24 hours), which was then lyophilized for 48 hours, and the images were taken using SEM.

Determination of Entrapment and Loading efficiencies: Around 5-10 mg of the particles was dissolved in methylene chloride and was vortexed for 30-50 seconds. The particles were then centrifuged at 10,000 rpm for 5-10 minutes. The supernatant was analyzed for Cy5.5.

Intraductal Retention Study

Female SD rats were anesthetized using isoflurane and the hair around the nipple region was removed using hair removing cream. The keratin plug from the inguinal mammary gland (5th or 6th nipple from the head) was removed gently holding the nipple using tweezers and wiping with 70% alcohol.

For microspheres and nanoparticles, around 0.5 to 1.5 mg particles was dispersed in PBS containing 0.5% w/v Tween 80 and vortexed for 30-60 seconds. For In situ implants, 50-100 μl of gel containing an equivalent amount of Cy5.5 was used. After dilation of the nipple orifice, 50-100 μl of the formulation was injected intraductally into the inguinal mammary glands using 27-31 G needle under a surgical microscope.

The animals were imaged using Bruker in Vivo Xtreme II optical imaging system at predetermined time points (0 to 96 hours) to study the distribution of the carrier system in the breast. Following instrument settings were used: Excitation/Emission: 690/750 nm; Bin: 1×1 pixels; Exposure time: 20 seconds, f-stop: 1.2, FOV: 19. Fluorescence intensities were plotted against time by choosing a constant ROI around the injected mammary gland and subtracting the fluorescence intensity from the contralateral mammary gland.

Intraductal Injection of Long Acting Carrier Systems

The carrier systems was admixed with crystal violet/Cy 5.5 and administered by intraductal injection. The rats were euthanized by CO₂ asphyxiation 15-30 minutes after injection. The animal was pinned to a dissection board. An excision was made in the abdomen area and the skin along with the mammary gland was gently detached from the underlying tissues using surgical scissors and a photograph was taken.

Mammary Whole Mounts

After 15-30 minutes of intraductal injections, rat was euthanized and the mammary whole mount was prepared using a previously established method with slight modifications. Briefly, mammary glands were dissected and mounted onto a glass slide and fixed in chloroform/isopropanol/acetic acid (6:3:1) for 6 hours. The glands were then defatted using acetone for 2-3 hours. The slides were then stored in methyl salicylate and fluorescent images were photographed with confocal microscopy using 20× objective.

Fluorescence Microscopy

At the end of the study (96 hours), the rat was euthanized, and mammary gland was excised. The tissue was washed with PBS and dried using kim wipe, snap frozen in OCT. The OCT block was then sectioned to 8-10 μm thick sections in a cryomicrotome at −25° C. The sections were observed using a fluorescence microscope under 20× objective. For nanoparticles, the procedure described above was used, except that the breast tissue was imaged after 48 hours. Cy5.5 was used as a control and the tissue was processed as described above and imaged after 4 hours.

Non-Invasive Imaging of Regional Lymph Nodes

Intraductal injection was performed following the method described in the previous section. Regional lymph nodes (Axillary lymph node) were identified and dissected at 2 hours, 24 hours and 48 hours after intraductal injection. The lymph nodes were then imaged using whole body animal imaging system to determine the localization of formulation in the lymph nodes.

Histology

Intraductal injection was performed as mentioned in the previous section. At the end of study (7 days) rats were euthanized and the mammary glands injected of different treatment groups were fixed in 10% formalin for 24 hours. The fixed glands were then embedded in paraffin wax and 5-10 μm sections were taken using microtome. The sections were then viewed under a stereo microscope under 20× objective lens.

Results

FIG. 3 shows the physicochemical characteristics of long acting PLGA formulations.

Scanning Electron Microscopy of PLGA/PDLLA Microspheres

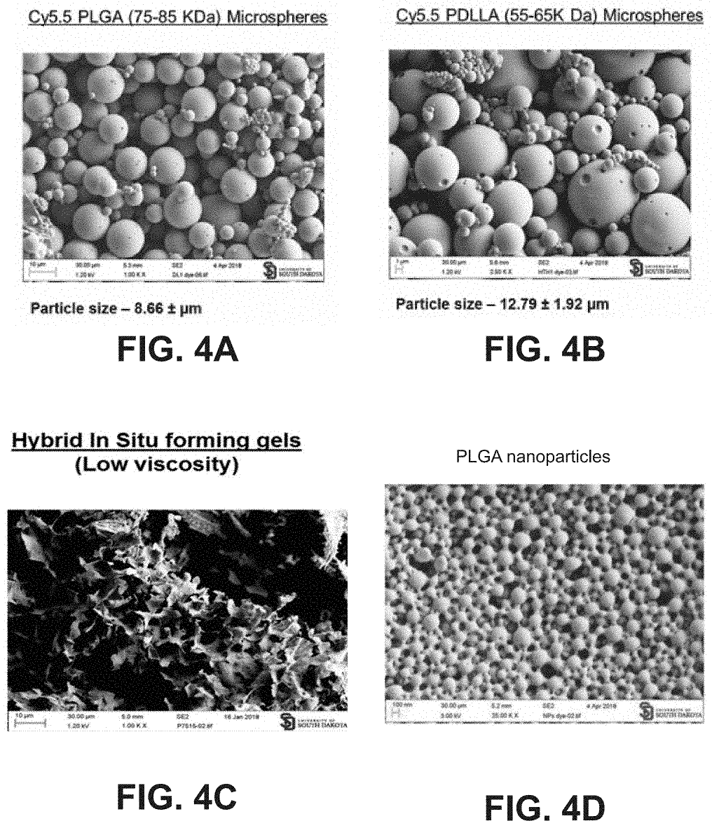

FIG. 4 shows representative Scanning Electron Microscopy (SEM) images of PLGA formulations (Microspheres and PLGA In situ forming implants).

In Vitro Release Profile

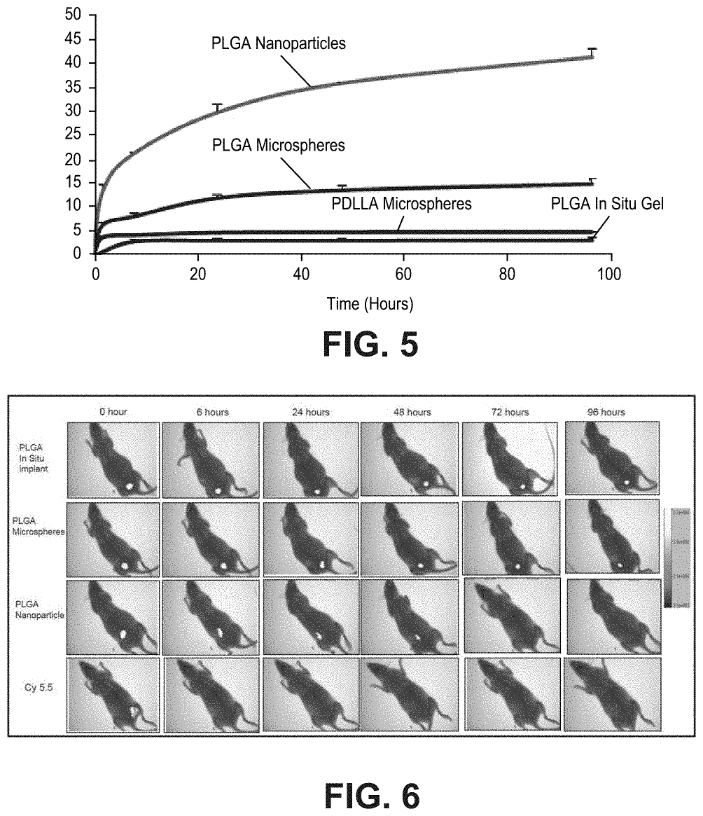

FIG. 5 shows In Vitro release profiles of microspheres (PLGA and PDLLA), nanoparticles and In situ forming implants (0-96 hours). Data=Mean±SD, n=3.

Intraductal Retention of Formulations Using Whole Body Animal Imaging System

FIG. 6 shows representative fluorescence images showing intraductal retention of different formulations captured using Bruker In Vivo Xtreme II whole body imaging system.

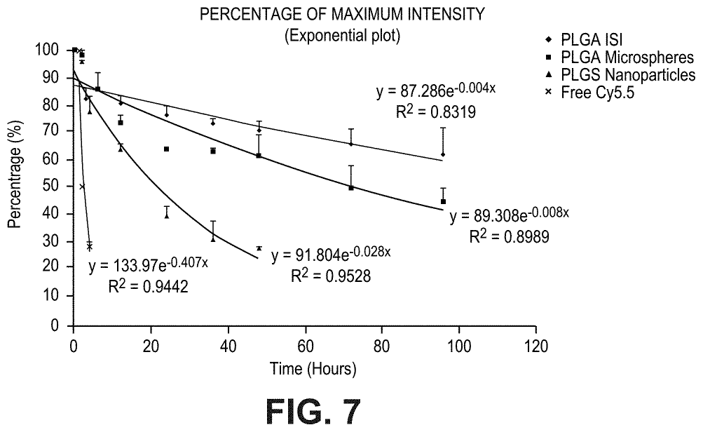

FIG. 7 shows fluorescence intensity profile expressed as percentage of maximum fluorescence intensity. Data point=Mean±SEM, n=3. The retention half-life is shown in the table below.

| Formulation | k (h⁻¹) | t_{1/2} (hrs) |
|---|---|---|
| PLGA In situ implants | 0.004 ± 0.002 | 192.5 ± 11.11 |
| PLGA Microspheres | 0.008 ± 0.002 | 88.35 ± 8.76 |
| PLGA nanoparticles | 0.02 ± 0.001 | 24.23 ± 1.12 |
| Cy 5.5 | 0.40 ± 0.03 | 1.70 ± 0.16 |

Intraductal Injection of PLGA Formulations

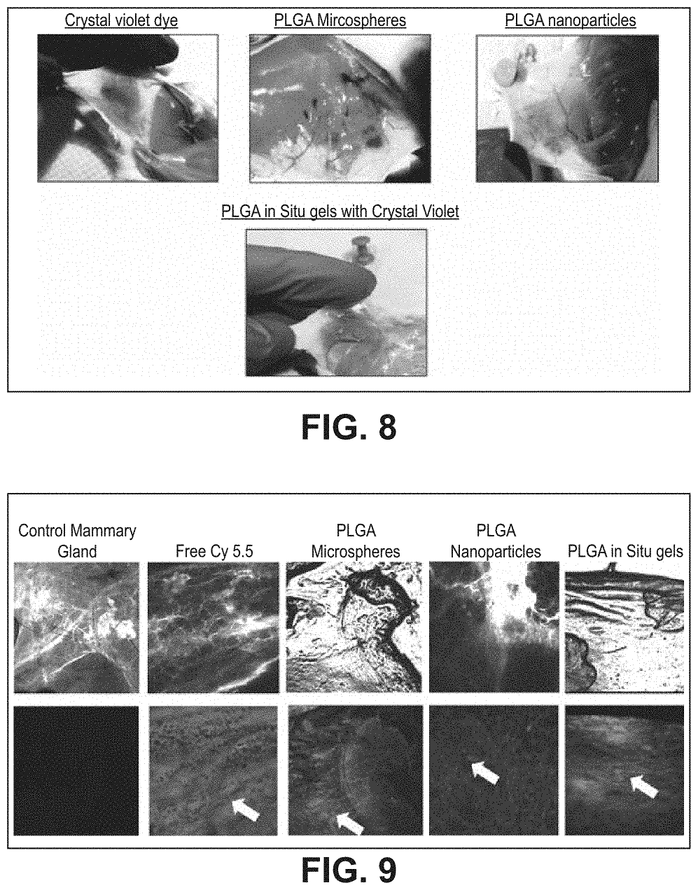

FIG. 8 shows photographs confirming intraductal localization of PLGA formulations using crystal violet.

Mammary Whole Mount of Long Acting Formulations

FIG. 9 shows mammary whole mounts showing the localization of formulations in the breast ducts. The panel on the top represents phase contrast images and the corresponding fluorescence images are shown at the bottom. The arrow in the inset indicate the particles retained within the breast ducts.

Fluorescence Microscopy

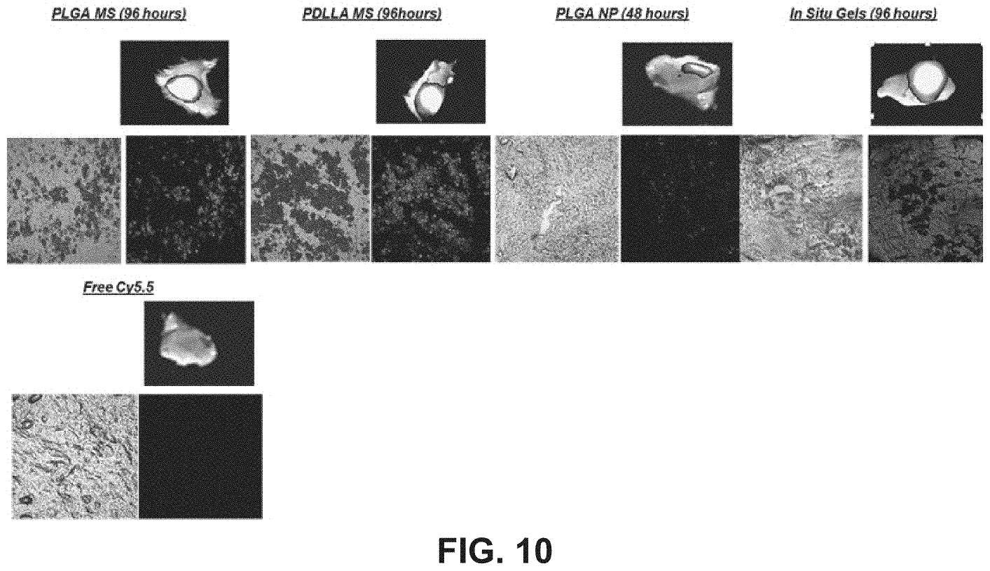

FIG. 10 shows fluorescence image of the excised mammary glands at 96 hrs (top panel) and the corresponding brightfield and fluorescence images. The images were captured using confocal microscopy under 20× objective.

Role of Regional Lymph Nodes in the Clearance of PLGA Formulations from Ducts

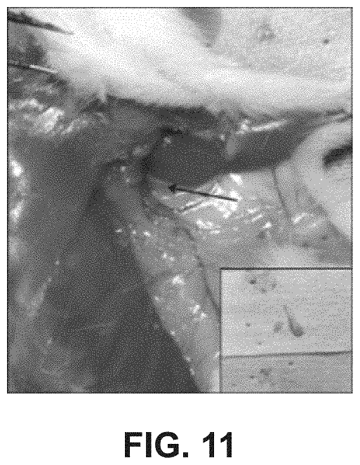

FIG. 11 shows an exemplary image of the axillary lymph node in female sprague dawley rats.

Localization of PLGA Formulations in Axillary Lymph Nodes Results

The emulsion solvent evaporation method using homogenization resulted in microspheres of particle sizes ranging from 8 to 15 μm. The particle sizes of nanoparticles were 200±17.08 nm. Entrapment and loading of Cy5.5 was dependent on particle size. Larger microspheres were able to entrap more Cy5.5 when compared to nanoparticles. Scanning electron microscopy studies showed spherical morphology of microspheres. PLGA in situ implant formation was confirmed using SEM. In Vitro release profiles showed sustained release of Cy5.5 from microspheres and in situ forming implants for 96 hours. Nanoparticles showed higher burst release and released Cy5.5 much faster than microspheres and in situ implants. This can be explained by the larger diffusion path length which the dye has to traverse in case of microspheres and gels which resulted in slower release. The in situ implants showed slowest release profile and released less than 10% of loaded Cy5.5 in 4 days. This can be explained by slow diffusion of drug from the much thicker implant matrix.

Consistent with our earlier observations with polystyerene particulate systems, microspheres were retained in the ducts for up to 96 hours in comparison to nanoparticles. PLGA In situ forming implants showed the highest retention in ducts. This demonstrates that the formulation matrix has a significant impact on intraductal retention, in addition to particle size.

Non-invasive retention studies were performed to determine the disposition of formulations from in the breast ducts With in-situ forming PLGA implants and PLGA microspheres (>1 μm), strong fluorescence signals were recorded up to 4 days. Nanoparticles (200 nm) were found to diffuse out of the ducts after 48 hours and no fluorescence signals were recorded for free Cy5.5 after 4 hours. The decrease in the fluorescence intensities were due to the diffusion of the formulation from the ducts. In situ forming implant showed highest fluorescence intensities, which confirms the formation of an implant in the ducts and slow release of the dye from the formulation matrix consistent with the in-vitro release profiles. PDDLA microspheres showed higher fluorescence intensities when compared to PLGA microspheres with similar particle sizes. This can be attributed due to the increased hydrophobicity of the PDLLA matrix compared to PLGA.

Images from mammary whole mounts (FIGS. 8 and 9) show the uniform distribution of formulation within the treated breast ducts. Fluorescence microscopy images (FIG. 10) confirms the retention of the formulations in the breast. PLGA/PDLLA microspheres and In situ forming implants showed bright fluorescence in the mammary gland, which was consistent with the in vivo imaging study, and no fluorescence was observed with nanoparticles and the free dye.

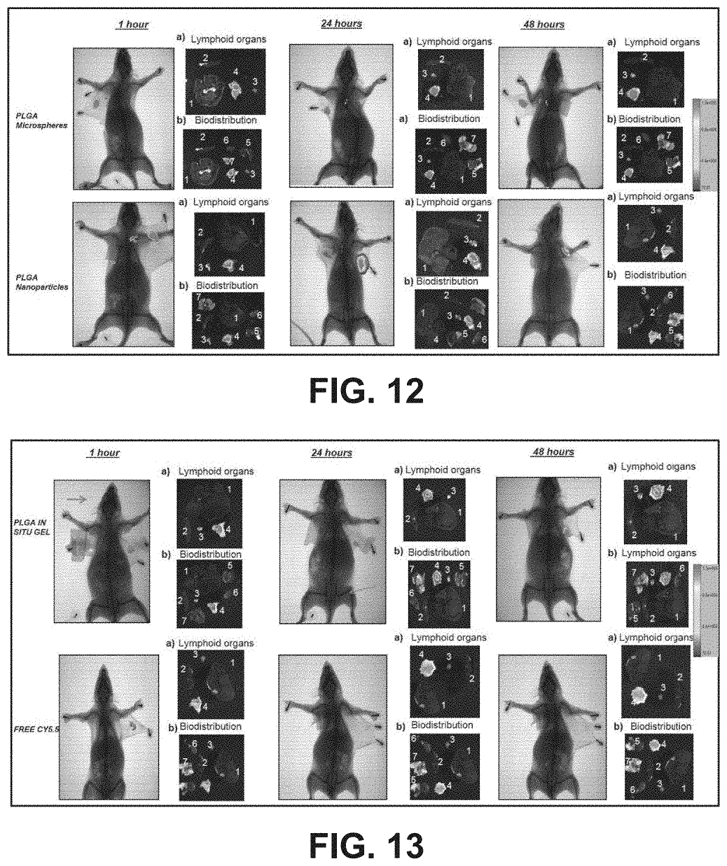

We further investigated the disposition of the formulations to regional lymph nodes (FIGS. 11-13). The results from the study indicated that microspheres were found to localize in the axillary lymph nodes up to 48 hours, in comparison to 24 hours with nanoparticles. Free Cy5.5 did not show any fluorescence in the lymph nodes beyond 1 hour. The results demonstrate that the distribution and retention in the regional lymph nodes is strongly dependent on the particle size. Free dye showed minimal distribution and retention in the regional lymph nodes.

Results from the histology studies (FIG. 14) showed no significant changes in the mammary glands treated with the formulations when compared to control mammary glands.

Intraductal Injection in Pig Model

Porcine Mammary Glands (6 Pairs were Used for Treatment)

Methodology

To confirm the findings from the rat studies in an animal model that is close to humans, pig was used. Five to six-month old female pig (gilt) was obtained from SDSU swine education and research facility and housed individually in pens. The pig was fed a standard finisher diet for one week prior to the commencement of the study. Before starting the treatment, the pig was anesthetized by an intramuscular injection of TKX (telazol and xylazine at 50 mg/mL each; ketamine at 100 mg/mL) using a 16-gauge sterile (1-inch-long) butterfly needle at 2.5 ml/50 kg body weight. The keratin plugs from the teats were removed by gently wiping the nipple surface with 70% alcohol. The formulations (500-1000 μL) were injected into thoracic, abdominal and inguinal mammary glands using 23 G blunt end needle under anesthesia. After intraductal injection, the animal was returned to the pen and housed for 4 days. At the end of the study, the animal was euthanized by an intravenous injection of pentobarbital-based euthanasia (1 mL/10 lb) using a butterfly needle to collect organs for further analysis. The mammary glands were imaged using Bruker In vivo imager.

Intraductal Retention of Formulations in Pig Breast Tissue Results

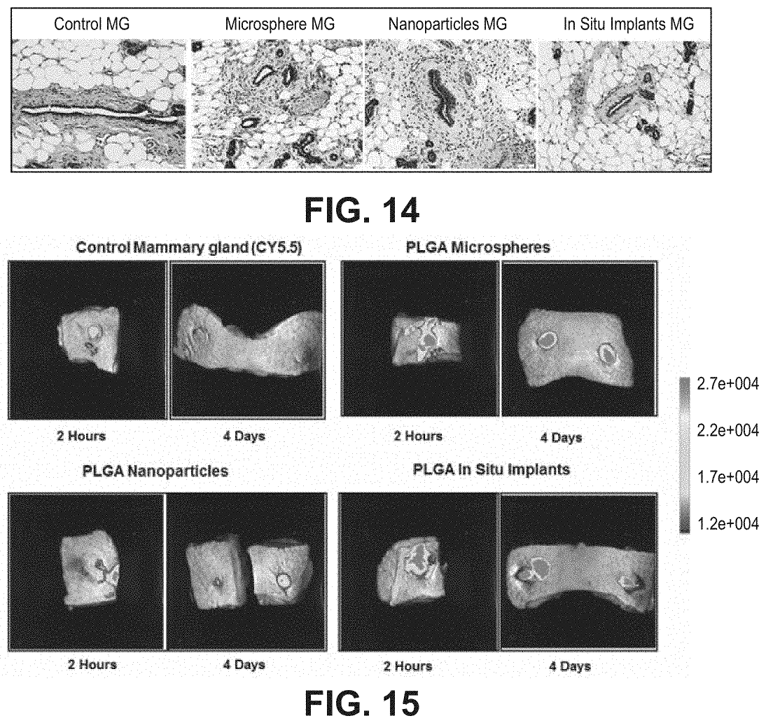

The results from the imaging studies in pig breast tissue (best seen in FIG. 15) were consistent with our previous findings in rats. The free dye diffused out of the mammary ducts within 2 hours. However, PLGA microspheres and In situ gel implants were retained in the breast for 4 days. On the other hand, with PLGA nanoparticles, the fluorescence intensity was significantly lower than the other two formulations. From the results, we can conclude that particle size and formulation matrix plays a significant role in formulation retention in the breast.

Development and Optimization of Tamoxifen (TMX) PLGA/PDLLA Formulations

Aim: Based on the promising results from the dye studies, drug formulations were developed. Our aim was to develop and optimize long acting PLGA/PDDLA microspheres, PLGA nanoparticles and PLGA in-situ gel formulations of tamoxifen (breast cancer drug).

Methodology

Formulation of Microspheres

Microspheres were prepared by oil-in-water (O/W) emulsion method. The required amounts of polymer and Tamoxifen (TMX; 50 mg) were dissolved in 5 ml dichloromethane (DCM). The organic phase was then added drop wise into an aqueous phase containing a surfactant (PVA). The mixture was then homogenized at 10,000 rpm for 1 minute/overhead stirring at 1000 rpm for 10 minutes to form microspheres. The emulsion was then stirred at 300-400 rpm for 3 hours to completely remove DCM. The hardened microspheres were then collected by centrifugation at 4000 rpm for 20 minutes. The pellet was washed, freeze dried, lyophilized, and stored at 4° C. and the lyophilized preparation was then used for further studies.

Formulation of Nanoparticles

PLGA nanoparticles were prepared by emulsion solvent evaporation by sonication method. Briefly, 100-200 mg of PLGA was dissolved in methylene chloride containing 10-50 mg TMX. The organic phase was then added drop wise into an aqueous phase containing 1-2% w/v PVA. The mixture was then sonicated using a probe sonicator set at 50 W of energy output for 1 minute in an ice bath to form oil-in-water (o/w) emulsion. The emulsion was then stirred at 300-400 rpm for 2 hours to remove residual organic solvent. The nanoparticles were separated by ultracentrifugation at 20,000 rpm for 20 minutes. The pellet was collected, washed and freeze dried and the lyophilized powder was stored at 4° C. for further use.

Formulation of In Situ Forming Implant

PLGA in situ forming gel was prepared by dissolving the polymer in a highly water miscible solvent like N-methyl-2-pyrrolidone (NMP). Briefly, 15 wt % PLGA was used to formulate in situ forming gels. In this study, PLGA of two copolymer (LA: GA) ratios, 50:50 and 75:25 of molecular weights 5-10 KDa and 10-15 KDa respectively were used to formulate the implants with desired characteristics for intraductal injection. TMX in NMP was dispersed into the polymer phase and dissolved by bath sonication (5 minutes) and was used for further studies. PLGA of different molecular weights were blended together to obtain the required viscosities and release profiles.

In Vitro Release Studies

Briefly 5-10 mg of microspheres and nanoparticles were dispersed in 1 to 2 ml of release medium (PBS, pH 7.4 containing 0.5% w/v SLS) in an Eppendorf tube. The tubes were then placed in an incubator shaker set at 100 rpm. At each time point, the tubes were centrifuged (10,000 rpm) and the release medium was collected and replaced with same volume of fresh medium to maintain sink condition. The supernatant was then analyzed using HPLC to determine the TMX concentrations in the release medium. For in-situ forming implants, 10 μg equivalent of gel was placed in 1 ml of release medium and the release study was carried out.

Results

A) Formulation Optimization of PLGA Microspheres

i) Effect of Preparation Method on Particle Characteristics and In Vitro Release of TMX from PLGA (10-15 KDa) Microspheres

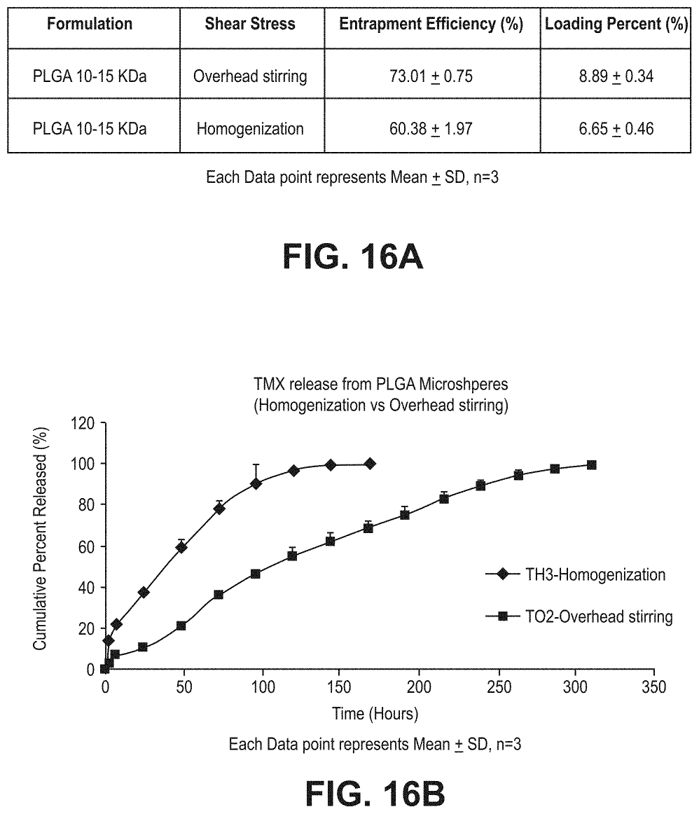

In this study PLGA (10-15 KDa) was used to formulate microspheres. TMX formulations with desired release profiles were prepared using homogenization (10,000 rpm for 60 seconds) and overhead stirring methods. Homogenization results in high shear and produced smaller microspheres compared to overhead stirring method. The results from the figure (FIG. 16) show the influence of particle size on TMX release from particles produced by homogenization and overhead stirring methods. Particles formed by stirring method showed higher entrapment efficiency (73.01±0.75) possibly due to higher particle size and lower burst release and sustained TMX release for 12 days.

ii) Effect of Preparation Method on Particle Characteristics and In Vitro Release of TMX from PLGA (75-85 KDa) Microspheres

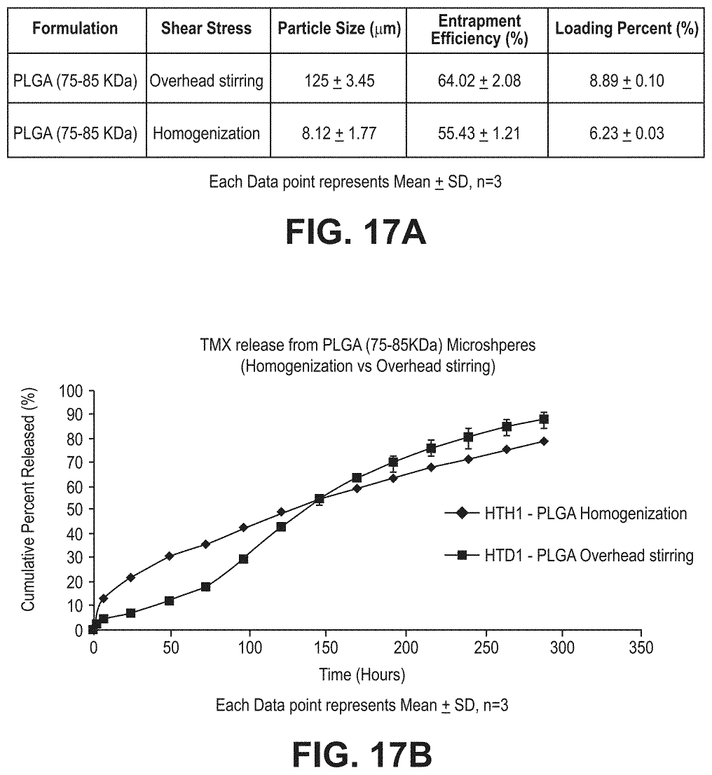

Higher molecular weight PLGA (75-85 KDa) was employed in the above study (FIG. 17) with the goal of producing sustained release formulations. Overhead stirring resulted in particle sizes >100 μm and was not found to be suitable for intraductal injection. However homogenization produced particles of smaller sizes (<10 μm) with a more sustained release profile when compared to microspheres prepared using lower molecular weight PLGA microspheres.

B) Formulation Optimization of PDLLA Microspheres

i) Impact of Preparation Method on PDLLA 55-65 KDa Microspheres

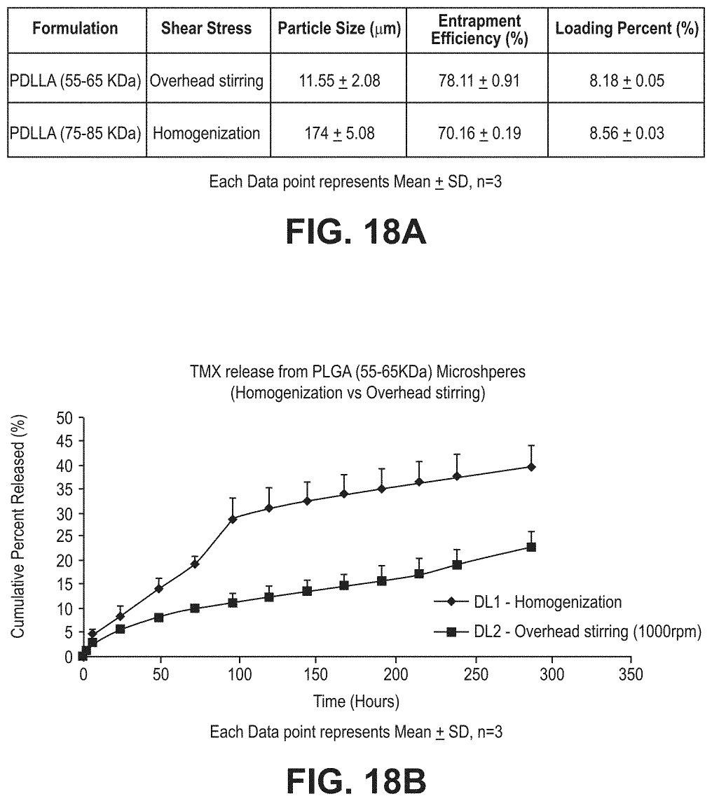

PDLLA, a more hydrophobic polymer was used in the above study (FIG. 18). Consistent with the results from high molecular weight PLGA microspheres, homogenization (10,000 rpm for 60 seconds) resulted in smaller microspheres. PDLLA microspheres with particle sizes <10 μm exhibited a more sustained TMX release (40% cumulative release in 12 days) in comparison to high molecular weight PLGA microspheres.

ii) Comparison of PLGA/PDLLA Microspheres with Different Particle Sizes and its Impact on In Vitro Release

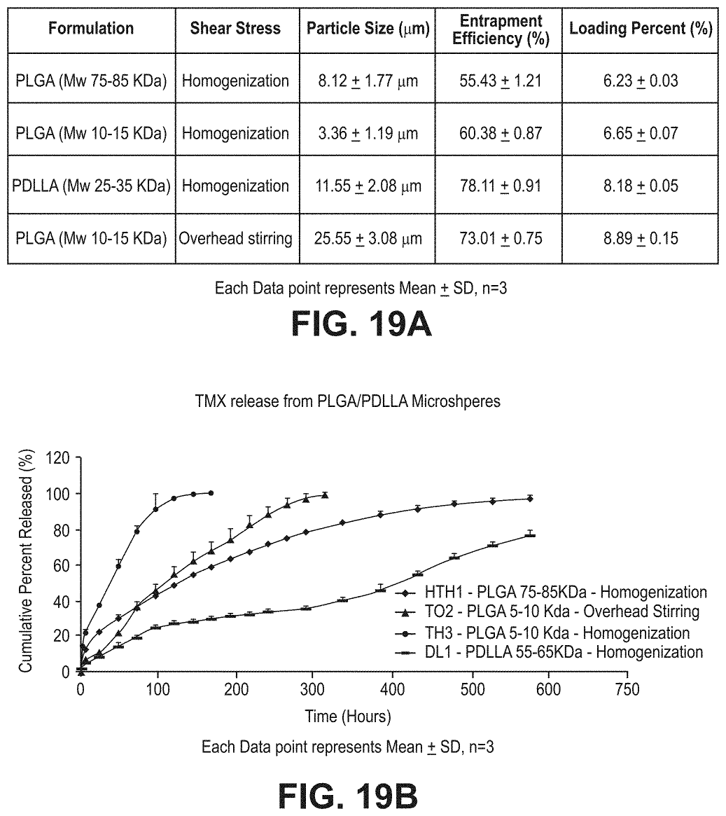

In the present study (FIG. 19), we used a combination of molecular weight and formulation method to obtain microspheres with desired particle size (10-15 μm) and sustained drug release profiles. Different PLGA and PDLLA microsphere formulations with drug release durations (100-600 hours) were formulated using the homogenization and overhead stirring methods as described in the previous sections. Higher molecular weight PLGA and PDLLA (HTH1 and DL1 respectively) in the particles size range of 10-15 μm sustained drug release >500 hours w. PLGA microspheres from lower molecular weight (10-15 KDa) released 100% of TMX in less than 2 weeks. Larger microspheres (25.55±3.08 μm) from lower molecular weight PLGA (TO2), showed a more sustained drug release (>250 hours). Particle size and molecular weight were found to have an impact on TMX release from microspheres.

C) Formulation Optimization of PLGA Nanoparticles

i) Influence of Drug Polymer Ratio on Tamoxifen PLGA Nanoparticles

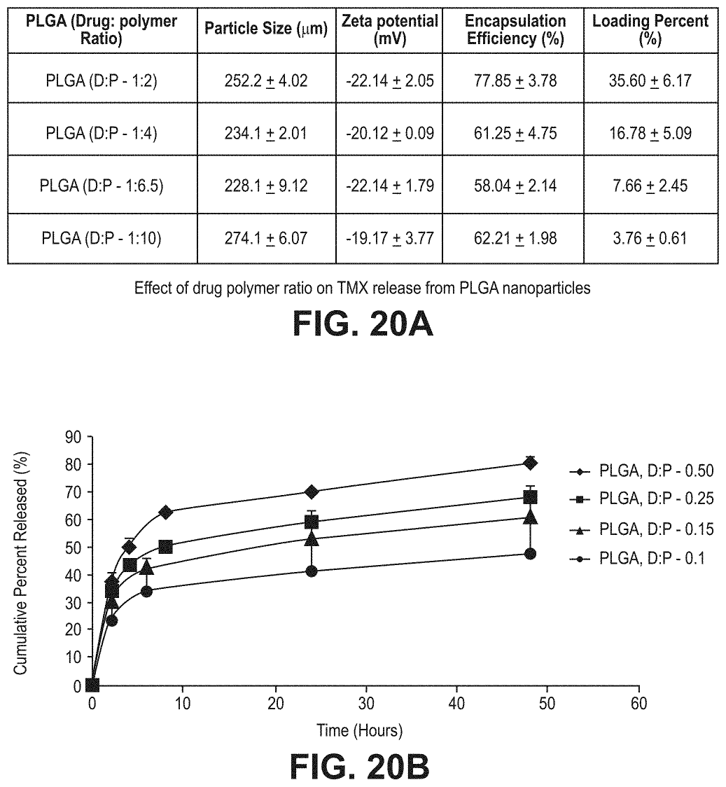

The goal of the study (FIG. 20) was to test the influence of drug polymer ratio on TMX release from nanoparticles. Drug polymer ratio was found to have an impact on the burst release of TMX from nanoparticles. PLGA nanoparticles with desired release profile and minimal burst release was achieved with drug polymer ratio 0.1 and hence was chosen for further studies.

D) Formulation Optimization of PLGA In Situ Forming Implant

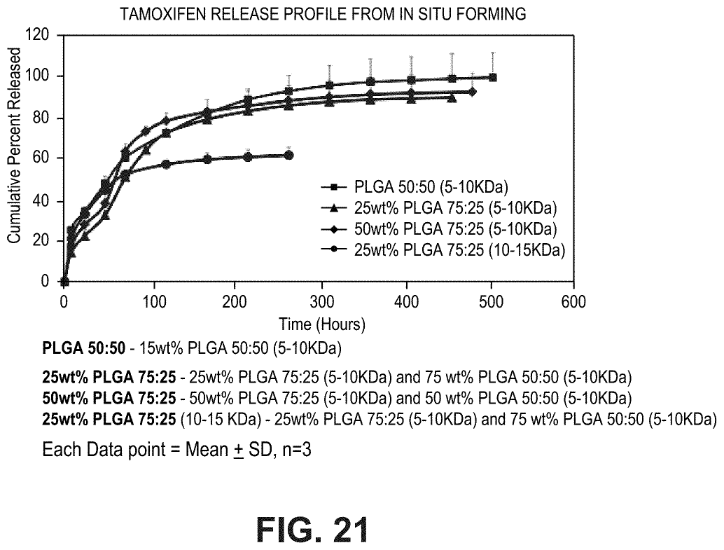

Viscosity is an important parameter to be considered for intraductal injections. In situ implants formed using low molecular weight PLGA (5-10 KDa) was found to be the least viscous, but showed higher burst release (FIG. 21). In situ implants with optimal viscosities were formulated using polymer blends of PLGA 50:50 (10-15 KDa and 5-10 KDa) and PLGA 75:25 (5-10 KDa) which were mixed at different weight ratios to form 15% PLGA in-situ gel. The results from the study demonstrated that blending PLGA 75:25 (10-15 KDa) at 25 wt % with PLGA 50:50 (5-10 KDa) resulted in reduction of TMX burst release, while maintaining desired viscosity.

In Vitro Formulation Optimization of Tamoxifen Formulations

Formulation of PLGA Poly (lactic-co-glycolic acid) microspheres: Poly (lactic-co-glycolic acid) (PLGA) of molecular weight 10-15 KDa and 75-85 KDa was used in the study. Required amount of tamoxifen and polymer was mixed with methylene chloride to form the organic phase. The organic phase was then added to aqueous phase containing 1% Poly (vinyl alcohol) (PVA) under magnetic stirring. The mixture was then either homogenized at 10,000 rpm for 1 minute or overhead stirring at 1000 rpm for 10 minutes. The formed emulsion was then magnetically stirred for 2-3 hours to remove organic solvent. The microspheres were collected by centrifugation and lyophilized for further use.

Methodology for in vitro release: 10 mg of microspheres/nanoparticles were dispersed in 2 ml release medium containing 0.05% w/v SDS (sodium dodecyl sulfate) as surfactant. The tubes were then placed in an incubator shaker at 100 rpm at 37° C. At each time points, the tube was centrifuged at 10,000 rpm for 10 minutes and the 1.8 ml of supernatant was analyzed for drug concentration using HPLC. The pellet was then gently redispersed using same volume of fresh release medium to maintain sink conditions and was placed back on the incubator shaker.

TABLE 1
Formulation parameters of PLGA microspheres

| | Particle size | Zeta Potential | | EE | LE |
|---|---|---|---|---|---|
| Formulation | (μm) | (mV) | PDI | (%) | (%) |
| PLGA 75:25 | 3.36 ± 1.19 | −31.36 ± 0.92 | 0.44 ± 0.09 | 55.43 ± 1.95 | 6.23 ± 0.14 |
| (10-15 KDa, H) | | | | | |
| PLGA 75:25 | 54.36 ± 12.93 | −30.48 ± 0.52 | 0.44 ± 0.09 | 73.01 ± 3.14 | 8.89 ± 0.26 |
| (10-15 KDa, OH) | | | | | |
| PLGA 75:25 | 9.12 ± 3.77 | −28.36 ± 0.74 | 0.44 ± 0.09 | 95.35 ± 2.01 | 10.22 ± 0.12 |
| (75-85 KDa, H) | | | | | |

PLGA is PLGA is poly (lactic-co-glycolic acid), LA is lactic acid, GA is Glycolic acid, H is homogenization, OH is overhead stirring. Each Value is Mean ± SD. Particle size calculated as an average of 30-50 microspheres using Smart Tiff software. EE is encapsulation efficiency, LE is loading efficiency, PDI is poly dispersity index

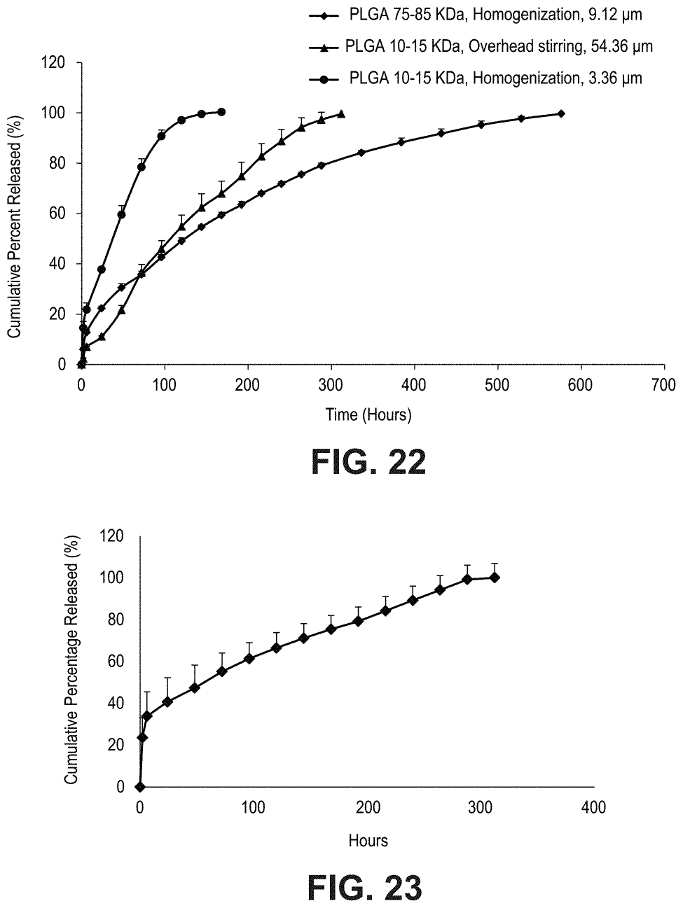

FIG. 22 shows the release of tamoxifen from formulations of different particle sizes formed using homogenization and overhead stirring. Each Value is Mean±SD.

Results: PLGA microspheres were formulated using emulsion solvent evaporation by homogenization/overhead stirring. The rationale of using the two methodologies for formulation and different molecular weight was to obtain smaller sizes microspheres with release profiles >14 days, which was the duration of in vivo study. PLGA of two different molecular weights (10-15 KDa and 75-85 KDa) were used in the study. With lower molecular weight PLGA 10-15 KDa, using homogenization and overhead stirring resulted in microspheres of particle size 3.36±1.19 and 54.36±12.93 μm respectively. However, the desired release profile was not attained with either of these formulations. With higher molecular weight PLGA (75-85 KDa), homogenization resulted in microspheres of particle size 9.12±3.77 μm with in-vitro drug release >14 days. Microspheres by homogenization formed spheres of smaller size compared to overhead stirring due to higher shear stress produced as a result of homogenization.

Formulation of Poly (lactic-co-glycolic acid) PLGA) nanoparticles: PLGA of molecular weight 10-15 KDa was used in the study. Required amount of tamoxifen and polymer was mixed with methylene chloride to form the organic phase. The organic phase was then added to aqueous phase containing 1% Poly (vinyl alcohol) (PVA) under magnetic stirring. The mixture was then sonicated at 50% amplitude using a probe sonicator for 1 minute to form the emulsion. The formed emulsion was then magnetically stirred for 2-3 hours to remove organic solvent. The nanoparticles were collected by ultracentrifugation and lyophilized for further use.

TABLE 2
Formulation parameters of PLGA Nanoparticles

| | Particle size | Zeta Potential | | EE | LE |
|---|---|---|---|---|---|
| Formulation | (nm) | (mV) | PDI | (%) | (%) |
| PLGA | 274.1 ± 4.87 | −19.17 ± 0.33 | 0.27 ± 0.024 | 91.28 ± 2.17 | 6.19 ± 0.17 |
| Nanoparticles | | | | | |

Each Value is Mean ± SD. EE is encapsulation efficiency, LE is loading efficiency, PDI is poly dispersity index

FIG. 23 shows the in vitro release profile of tamoxifen from optimized PLGA nanoparticles of particle size 274.1±4.87. Each Value is Mean±SD.

Results: PLGA nanoparticle was formulated using emulsion solvent evaporation by sonication. PLGA nanoparticles sustained tamoxifen release for >10 days. The formulations were optimized for optimal drug polymer ratio (data not shown) to sustain drug release. Optimized nanoparticles showed burst release of 33.84% and sustained tamoxifen release for >10 days.

Formulation of PLGA in-situ gel (ISG): PLGA of two different molecular weights 5-10 KDa and 10-15 KDa was used in the study. Two polymers were mixed in equal weight ratios (50:50 w/w) to form 25 wt % PLGA ISG. N-methyl pyrrolidone (NMP) was used as the solvent.

Methodology for in vitro release: 500 μl of ISG was injected into 4 ml of release medium contained in a scintillation vial containing 0.05% w/v of SDS to form the gel. The vial was then placed in an incubator shaker at 100 rpm at 37° C. At each time points, 1 ml release medium was drawn from the vial and replaced with same volume of fresh release medium to maintain sink conditions. The drug concentration was determined using HPLC.

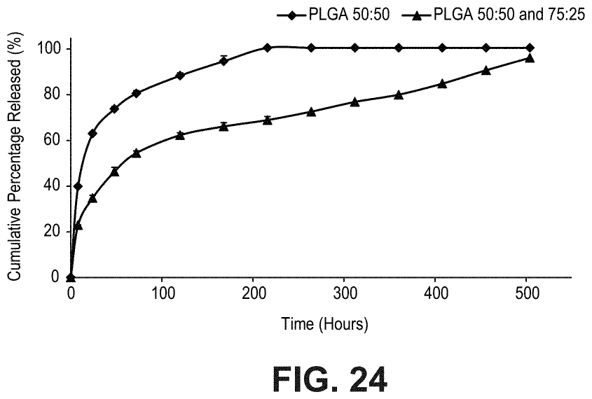

FIG. 24 shows the In Vitro release profile of tamoxifen from PLGA in situ gel. PLGA (LA:GA 50:50) (M_{w} 5-10 KDa) and PLGA (LA:GA 75:25) (M_{w} 10-15 KDa). Each Value is Mean±SD.

Results: Lower molecular weight polymer (5-10 KDa) released tamoxifen in 200 hours. Blending higher molecular weight PLGA 75:25 (10-15 kDa) to the lower molecular weight polymer sustained tamoxifen release >20 days while maintaining injectability.

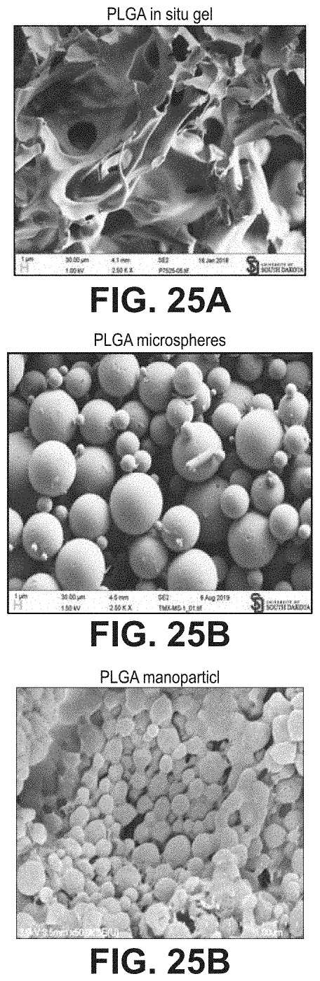

FIG. 25 shows Scanning Electron Microscope images of optimized formulations of PLGA in-situ gel, microspheres and nanoparticles.

In Vivo Study

Methodology

For In vivo studies, the levels of Tamoxifen and two major metabolites 4-hydroxytamoxifen (4HT) and endoxifen (EDX) were measured using LC-MS. (Liquid chromatography-Mass spectroscopy)

Plasma Extraction

For determining drug levels in plasma, 100 μl of plasma was mixed with 400 μl of acetonitrile to precipitate proteins (1:4 v/v). This was then vortexed for 1-2 minutes followed by sonication for 3 minutes. The plasma was then centrifuged at 10,000 rpm at 4° C. for 10 minutes. The supernatant was collected and evaporated to dryness under gentle stream of Argon. The residue was then reconstituted with 100 μl of ammonium formate, (pH 3.5): acetonitrile (7:3 v/v). This was vortexed for 1 minute followed by sonication for 2 minutes. The reconstituted mixture was centrifuged at 10,000 rpm for 10 minutes at 4° C. The supernatant was collected and 15 μl was injected into LC-MS to determine drug levels.

Organ Extraction

The mammary gland and all the other vital organs (liver, spleen, kidney, lungs, heart, and uterus) were weighed and washed with PBS to remove blood. The tissues were mixed with TRIS HCl buffer (1 ml/mg) and homogenized at 6000 rpm for 3 minutes under ice. For lymph nodes, sonication (45% amplitude in pulse mode 1 sec on and 1 sec off for 2 minutes under ice) was used for homogenization. From this, 200 μl of tissue homogenate was mixed with 800 μl of acetonitrile (1:4 v/v) for protein precipitation. This was vortexed for 1-2 minutes followed by sonication for 3 minutes. The homogenate was centrifuged at 10,000 rpm at 4° C. for 10 minutes. The supernatant was collected and evaporated to dryness under gentle stream of Argon. The residue was reconstituted with 100 μl of ammonium formate (pH 3.5): acetonitrile (7:3 v/v). This was vortexed for 1 minute followed by sonication for 2 minutes. The reconstituted mixture was centrifuged at 10,000 rpm for 10 minutes at 4° C. The supernatant was gently removed and 15 μl was injected into LC-MS for quantification.

TABLE 3
LCMS method (gradient table)

| | A | | |
|---|---|---|---|
| | (Ammonium Formate | (B) | |
| Time | 3.5 mM, pH 3.5 adjusted | 100% Acetonitrile | Flow Rate |
| (Mins) | with Formic acid) (%) | (%) | (ml/min) |
| 0 | 70 | 30 | 0.250 |
| 4 | 30 | 70 | 0.250 |
| 4.10 | 20 | 80 | 0.250 |
| 8 | 20 | 80 | 0.250 |
| 8.10 | 0 | 100 | 0.250 |
| 12 | 0 | 100 | 0.250 |
| 12.10 | 30 | 70 | 0.250 |
| 16 | 70 | 30 | 0.250 |
| 16.10 | 70 | 30 | 0.250 |
| 22 | 70 | 30 | 0.250 |

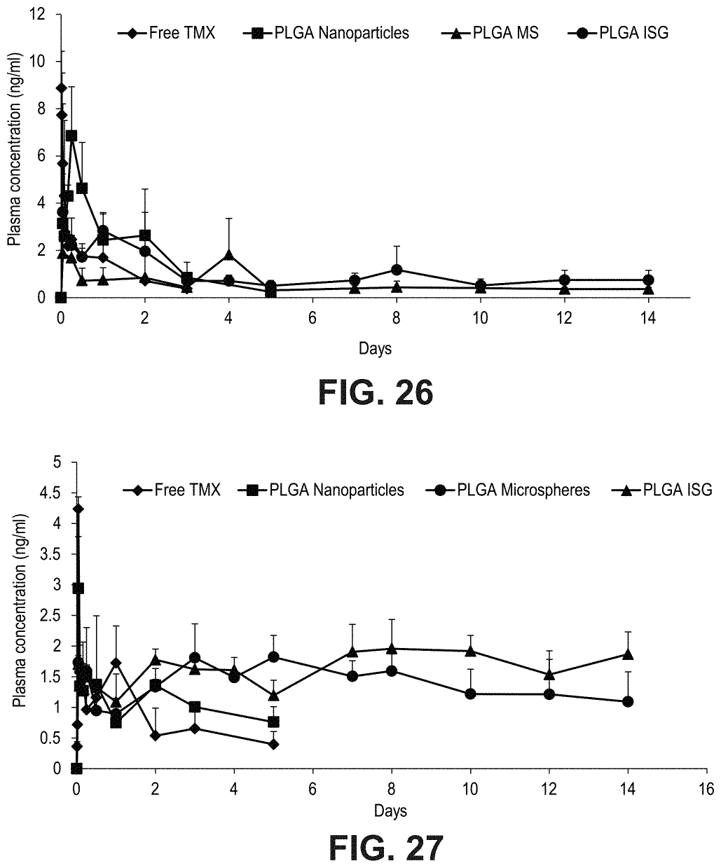

FIG. 26 shows the plasma profile of tamoxifen after intraductal injection of formulations. The plasma concentration was measured for 3 days for free tamoxifen, 5 days for PLGA nanoparticles and 14 days for PLGA microspheres (MS) and in situ gel. ISG is in situ gel. ISG is in-situ gel. Each value is Mean±SD, n=3

FIG. 27 shows the profile of 4-hydroxytamoxifen after intraductal injection of PLGA formulations. The plasma concentration was measured for 5 days for free tamoxifen, 5 days for PLGA nanoparticles and 14 days for PLGA microspheres and in situ gel. ISG is in situ gel. ISG is in situ gel. Each value is Mean±SD, n=3.

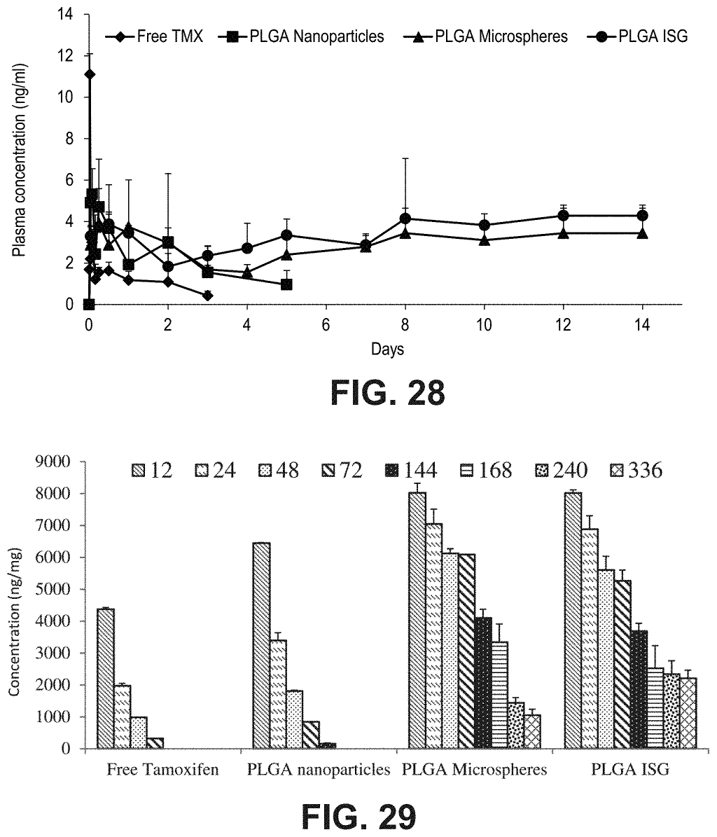

FIG. 28 shows the plasma profile of Endoxifen after intraductal injection of PLGA formulations. The plasma concentration was measured for 3 days for free tamoxifen, 5 days for PLGA nanoparticles and 14 days for PLGA microspheres and in situ gel. ISG is in situ gel. Each Value is Mean±SD, n=3.

Results: The systemic levels of tamoxifen and major metabolites were lower for PLGA MS and ISG. PLGA MS and ISG sustained drug levels up to 14 days but at lower concentrations (<5 ng/ml). Plasma level of endoxifen was highest when compared to tamoxifen and 4-hydroxytamoxifen in all treatment groups. Tamoxifen levels were measured in the plasma for 3 and 5 days respectively after which the levels were undetectable. Also, free tamoxifen and PLGA nanoparticles were not retained in the mammary glands after 3 and 6 days respectively (FIG. 8). Tamoxifen and metabolite levels were measured for 14 days for PLGA microspheres and in-situ gel.

Breast Concentration of Tamoxifen and Metabolites 4-Hydroxytamoxifen and Endoxifen

FIG. 29 shows the breast concentration of tamoxifen in the mammary glands at different time points (12, 24, 48, 72, 144, 168, 240 and 336 hours). ISG is in-situ gel. Each value is Mean±SD, n=3.

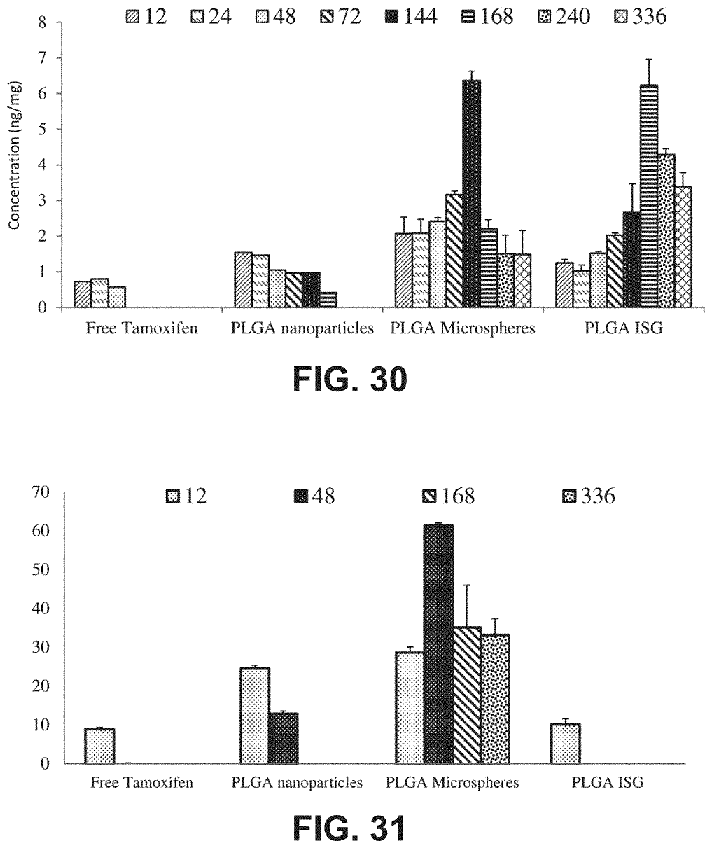

FIG. 30 shows the breast concentration of 4-hydroxytamoxifen at different time points (12, 24, 48, 72, 144, 168, 240 and 336 hours). Each value is Mean±SD, n=3.

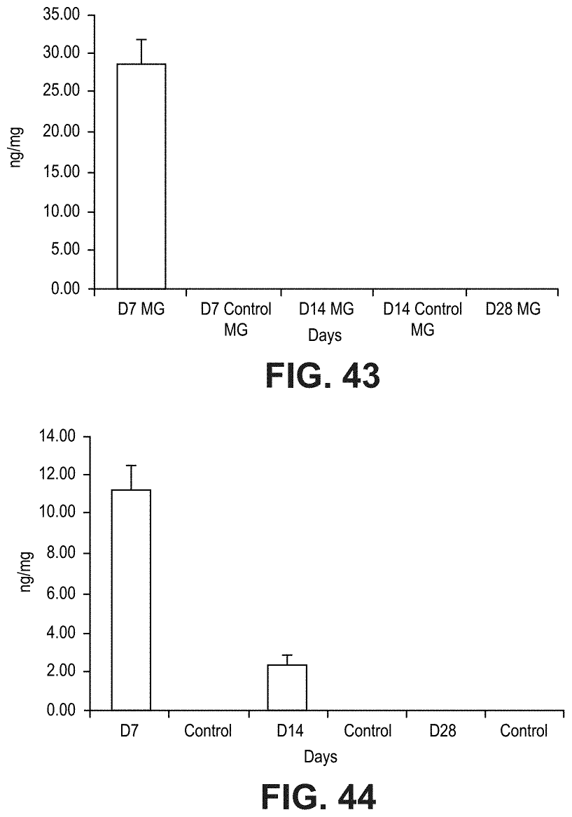

Results: PLGA MS and ISG were retained in the mammary gland for up to 14 days (FIG. 43) whereas free TMX and PLGA nanoparticles were retained for only 72 and 144 hours respectively. At day 14, the concentration of tamoxifen in the mammary gland was >1000 fold and >2000 fold higher for PLGA MS and ISG respectively compared to free tamoxifen and PLGA nanoparticles. Another major metabolite of tamoxifen, 4-hydroxytamoxifen was detected in the mammary glands. The level of 4HT in mammary glands treated with PLGA microspheres was 6-fold higher than PLGA nanoparticles and free tamoxifen at 144 hours. Same was the case for PLGA ISG. No 4HT was detected in free tamoxifen and PLGA nanoparticle group after day 3 and 7 respectively. At day 14, 4HT levels were 1.5 and 3 fold higher for PLGA microspheres and ISG compared to PLGA nanoparticles and free tamoxifen. Metabolites of tamoxifen were detected in the lymph nodes, but at much lower levels. This might be possibly coming from the metabolites in the blood that recirculates through lymph nodes.

Lymph Node Localization of PLGA Formulations

FIG. 31 shows lymph node concentration of Tamoxifen at different time points (12-336 hrs). Each Value is Mean±SD, n=3.

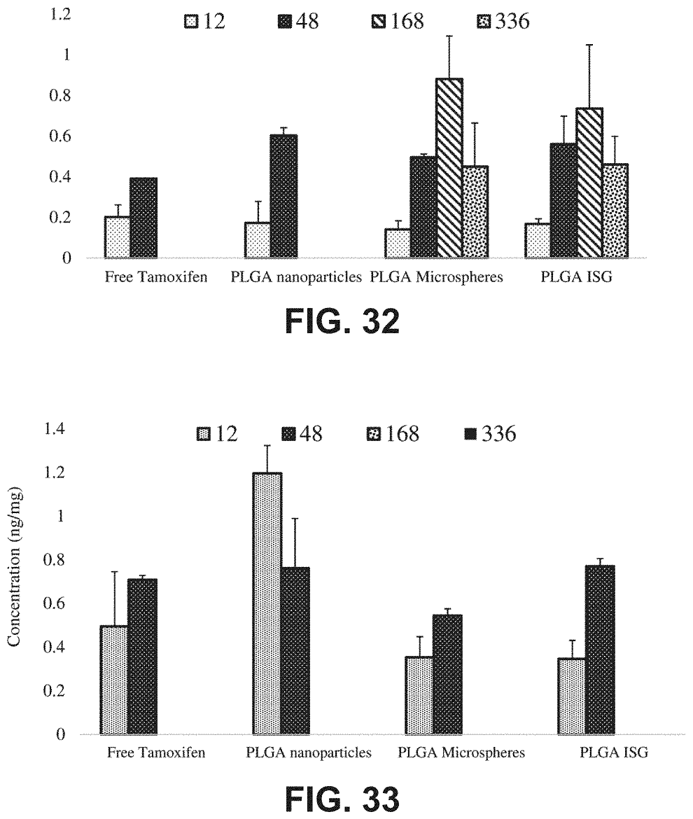

FIG. 32 shows the lymph node concentration of Endoxifen at different time points (12-336 hrs). Each Value is Mean±SD, n=3.

FIG. 33 shows the lymph node concentration of 4-Hydroxytamoxifen at different time points (12-336 hrs). Each Value is Mean±SD, n=3.

Results: PLGA microspheres showed higher levels of TMX in the lymph node up to 336 hours. At 336 hour, tamoxifen levels were >30 fold higher than free tamoxifen. PLGA ISG and free tamoxifen did not show lymph node localization of tamoxifen after 12 hours. PLGA nanoparticles was localized in the lymph node till 48 hours, but not detected at later time points.

Organ Distribution of PLGA Formulations

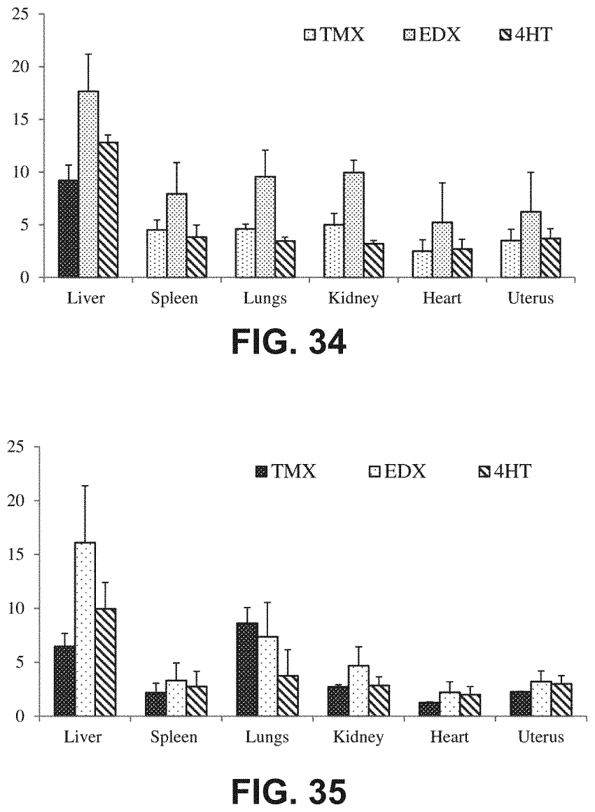

FIG. 34 shows the biodistribution of intraductal free tamoxifen at the end of the treatment. Each value is Mean±SD, n=3.

FIG. 35 shows the biodistribution of Intraductal PLGA Nanoparticles at the end of the treatment. Each Value is Mean±SD, n=3.

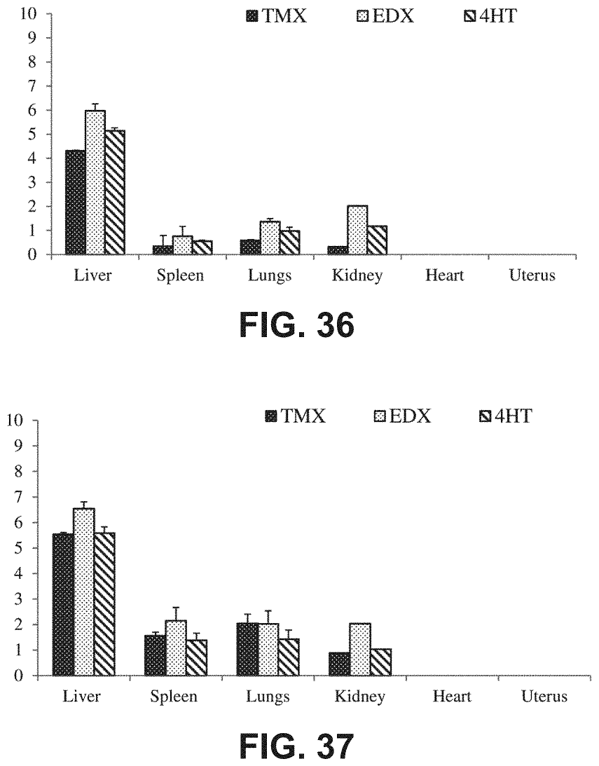

FIG. 36 shows the biodistribution of Intraductal PLGA Microspheres at the end of the treatment. Each Value is Mean±SD, n=3.

FIG. 37 shows the biodistribution of Intraductal PLGA in-situ gel at the end of the treatment. Each Value is Mean±SD, n=3.

Results: The systemic exposure of tamoxifen, endoxifen and 4-hydroxytamoxifen was lower in PLGA MS and ISG compared to PLGA nanoparticles and free tamoxifen. Liver being the major metabolizing organ showed higher drug levels amongst all organs. However, PLGA MS and ISG showed lower amounts in the liver in comparison to free tamoxifen and PLGA nanoparticles. Endoxifen levels were predominantly higher in comparison to the parent compound and 4-hydroxytamoxifen in all organs. Intraductal tamoxifen resulted in uterine exposure of tamoxifen and metabolites, but was at undetectable levels with PLGA MS and ISG.

TABLE 4
Pharmacokinetic parameters of Tamoxifen in plasma

| | t_{1/2} | kₑ | Cₘₐₓ | Tₘₐₓ | AUC | AUMC | MRT |
|---|---|---|---|---|---|---|---|
| | (hrs) | (hrs⁻¹) | (ng/ml) | (hrs) | (ng · hr/ml) | (ng · hr²/ml) | (hrs) |
| Free TMX | 19.51 ± 4.24 | 0.015 ± 0.002 | 8.9 ± 1.55 | 0.3 | 115.8 ± 43.31 | 4546.37 ± 466.103 | 47.7 ± 3.15 |
| Nano- | 56.75 ± 35.33 | 0.0066 ± 0.003 | 6.9 ± 2.1 | 8 ± 3.4 | 260.9 ± 35.41 | 12666.2 ± 466.10 | 47.7 ± 3.15 |
| particles | | | | | | | |
| Micro- | 92.89 ± 26.08 | 0.0033 ± 0.0009 | 2.9 ± 0.72 | 72 | 211.23 ± 89.43 | 34635.5 ± 25952.3 | 147.73 ± 59.28 |
| spheres | | | | | | | |
| ISG | 110.75 ± 31.15 | 0.0028 ± 0.0007 | 3.63 ± 0.15 | 80 ± 27.71 | 575.96 ± 88.14 | 136427 ± 76773.6 | 230.2 ± 0.68 |

t_{1/2}—plasma half-life; ke—elimination rate constant; Cmax—peak plasma concentration; Tmax—time to reach peak plasma concentration; AUC—area under the curve; AUMC—area under the first moment curve;; MRT—mean residence time. TMX—is tamoxifen; ISG is PLGA in-situ gel.

TABLE 5
Pharmacokinetic parameters of Endoxifen in plasma

| | t_{1/2} | kₑ | Cₘₐₓ | Tₘₐₓ | AUC | AUMC | MRT |
|---|---|---|---|---|---|---|---|
| | (hrs) | (hrs⁻¹) | (ng/ml) | (hrs) | (ng · hr/ml) | (ng · hr²/ml) | (hrs) |
| Free TMX | 34.19 ± 25.42 | 0.0142 ± 0.0127 | 11.1 ± 1 | 2 | 174.26 ± 19.38 | 8312.83 ± 2050.43 | 46.4 ± 5.95 |
| Nano- | 37.92 ± 6.43 | 0.0083 ± 0.0012 | 4.76 ± 0.26 | 1 | 219.5 ± 22.3 | 9967 ± 1628.2 | 44.9 ± 2.6 |
| particles | | | | | | | |
| Micro- | 247.27 ± 90.59 | 0.0013 ± 0.0003 | 5 ± 1.10 | 2.33 ± 0.88 | 2177.63 ± 580.41 | 1155923.16 ± 594100.43 | 472.56 ± 124.02 |
| spheres | | | | | | | |
| ISG | 292.30 ± 112.05 | 0.0013 ± 0.0003 | 5.36 ± 1.10 | 2 | 3125.06 ± 1050.41 | 2279641.4 ± 1447487.3 | 592.93 ± 205.86 |

t _{1/2}—plasma half-life; ke - elimination rate constant; Cmax - peak plasma concentration; Tmax- time to reach peak plasma concentration; AUC—area under the curve; AUMC—area under the first moment curve;; MRT—mean residence time. TMX—is tamoxifen; ISG is PLGA in-situ gel.

TABLE 6
Pharmacokinetic parameters of 4-hydroxytamoxifen in plasma

| | t_{1/2} | kₑ | Cₘₐₓ | Tₘₐₓ | AUC | AUMC | MRT |
|---|---|---|---|---|---|---|---|
| | (hrs) | (hrs⁻¹⁾ | (ng/ml) | (hrs) | (ng. hr/ml) | (ng. hr₂/ml) | (hrs) |
| Free TMX | 33.16 ± 20.20 | 0.0204 ± | 4.2 ± 0.115 | 1 | 92.56± | 4153.8 ± | 43.56 ± |
| | | 0.011 | | | 19.45 | 1094.60 | 3.19 |
| Nanoparticles | 38.51 ± 1.83 | 0.0077 ± | 3.1 ± 0.36 | 4.6 ± 3.6 | 168.1 ± 1.80 | 14150.3 ± | 84.03 ± |
| | | 0.0003 | | | | 1373.2 | 7.39 |
| Microspheres | 157.11 ± 26.29 | 0.002 ± | 2.36 ± 0.26 | 112 ± | 679.2 ± 169.94 | 188094.86 ± | 252.03 ± |
| | | 0.0004 | | 42.33 | | 77107.74 | 49.79 |
| ISG | 94.27 ± 23.52 | 0.0037± | 2.36 ± 0.17 | 224 ± | 813.06 ± 97.36 | 220136.53 ± | 264.66 ± |
| | | 0.0011 | | 42.33 | | 52211.86 | 32.21 |

t½ - plasma half-life; ke - elimination rate constant; Cmax - peak plasma concentration; Tmax - time to reach peak plasma concentration; AUC - area under the curve; AUMC - area under the first moment curve; MRT - mean residence time. TMX - is tamoxifen; ISG is PLGA in-situ gel.

TABLE 7
Pharmacokinetic parameters of tamoxifen in breast

| | t_{1/2} | kₑ | Cₘₐₓ | Tₘₐₓ | AUC |
|---|---|---|---|---|---|
| Free TMX | 14.87 ± 0.04 | 0.020 ± 0.0005 | 4373.8 ± 67.70 | 12 | 125355.4 ± 3692.96 |
| Nanoparticles | 28.61 ± 5.74 | 0.0105 ± 0.002 | 6436.3 ± 15.41 | 12 | 235444.4 ± 2744.84 |
| Microspheres | 204.805± | 0.00125± 0.0003 | 8080.7 ± 93.33 | 12 | 1493077.05 ± 33219.38 |
| ISG | 557.13 ± 52.93 | 0.0007 ± 0.0003 | 8282.6 ± 248.26 | 12 | 2916866.333 ± 197615.3 |

| | AUMC | MRT |
|---|---|---|
| Free TMX | 3654696 ± 120023.3 | 26.5 ± 4.3 |
| Nanoparticles | 9360274.65 ± 874274.5 | 39.7 ± 3.25 |
| Microspheres | 325087882.4 ± 28461186 | 217.55 ± 14.21 |
| ISG | 2116349830 ± 354466261.3 | 755 ± 108.64 |

t_{1/2}—plasma half-life; kₑ—elimination rate constant; Cₘₐₓ—peak plasma concentration; Tₘₐₓ—time to reach peak plasma concentration; AUC—area under the curve; AUMC—area under the first moment curve;; MRT—mean residence time. TMX—is tamoxifen; ISG is PLGA in-situ gel.

TABLE 8
Pharmacokinetic parameters of 4-hydroxytamoxifen in breast

| | t_{1/2} | kₑ | Cₘₐₓ | Tₘₐₓ | AUC | AUMC | MRT |
|---|---|---|---|---|---|---|---|
| Free | 49.06 ± 12.37 | 0.0064 ± 0.001 | 0.86 | 32 ± 13.85 | 88.33 ± 7.07 | 7689.96 ± 1836.07 | 86.53 ± 15.83 |
| TMX | | | | | | | |
| Nano- | 59.12 ± 23.84 | 0.005 ± 0.002 | 2.1 ± 0.70 | 90 ± 110.30 | 365.15 ± 171.19 | 61320.1 ± 47356.5 | 154.5 ± 57.27 |
| particles | | | | | | | |
| Micro- | 116.62 ± 5.64 | 0.002 | 6.25 ± 1.62 | 144 | 1118.35 ± 214.04 | 239585.9 ± 18355.8 | 216.6 ± 25.03 |
| spheres | | | | | | | |
| ISG | 319.34 ± 29.35 | 0.0009 ± 0.0001 | 6.23 ± 0.64 | 168 | 1959.73 ± 840.76 | 1365242.6 ± 485313 | 528.3 ± 67.64 |

t_{1/2}—elimination half-life from the breast; kₑ—elimination rate constant from the breast; Cₘₐₓ—peak brewt concentration; Tₘₐₓ—time to reach peak breast concentration; AUC—area under the curve; AUMC—area under the first moment curve;; MRT—mean residence time in the breast. TMX—is tamoxifen; ISG is PLGA in-situ gel.

TABLE 9
Pharmacokinetic parameters of 4-hydroxytamoxifen in breast

| | t_{1/2} | kₑ | Cₘₐₓ | Tₘₐₓ | AUC | AUMC | MRT |
|---|---|---|---|---|---|---|---|
| Free TMX | 49.06 ± 12.37 | 0.0064 ± 0.001 | 0.86 | 32 ± 13.85 | 88.33 ± 7.07 | 7689.96 ± 1836.07 | 86.53 ± 15.83 |
| | | 0.005 ± 0.002 | | | | | |
| Nanoparticles | 59.12 ± 23.84 | | 2.1 ± 0.70 | 90 ± 110.30 | 365.15 ± 171.19 | 61320.1 ± 47356.5 | 154.5 ± 57.27 |
| Microspheres | 116.62 ± 5.64 | 0.002 | 6.25 ± 1.62 | 144 | 1118.35 ± 214.04 | 239585.9 ± 18355.8 | 216.6 ± 25.03 |
| ISG | 319.34 ± 29.35 | 0.0009 ± 0.0001 | 6.23 ± 0.64 | 168 | 1959.73 ± 840.76 | 1365242.6 ± 485313 | 528.3 ± 67.64 |

t_{1/2}—elimination half-life from the breast; kₑ—elimination rate constant from the breast; Cₘₐₓ—peak brewt concentration; Tₘₐₓ—time to reach peak breast concentration; AUC—area under the curve; AUMC—area under the first moment curve;; MRT—mean residence time in the breast. TMX—is tamoxifen; ISG is PLGA in-situ gel.

Formulation Optimization of 4-Hydroxytamoxifen Formulations

Formulation Parameters of 4-Hydroxytamoxifen (4Ht) Loaded PLGA Microspheres

| | Particle | Encapsulation | Loading |
|---|---|---|---|
| Formulation | size (μm) | Efficiency (%) | Efficiency (%) |
| PLGA Microspheres | 9.63 ± 1.49 | 92.46 ± 2.59 | 10.46 ± 0.38 |
| (LA:GA-75:25), | | | |
| Mw - 75-85 KDa | | | |

PLGA is poly (lactic-co-glycolic acid), LA is lactic acid, GA is Glycolic acid, Mw is molecular weight. Each value represents Mean ± S.D.

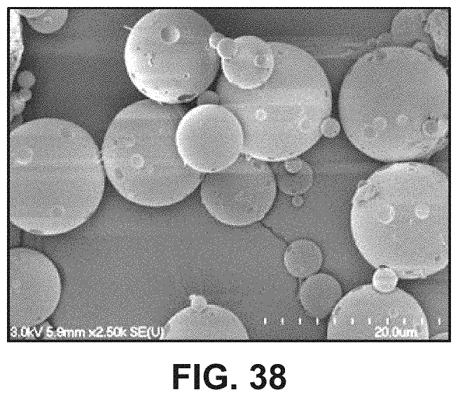

FIG. 38 shows a scanning electron microscopy image of 4HT loaded PLGA microspheres.

Formulation Optimization of PCLA-PEG-PCLA Thermogel

The optimized percentage of PCLA-PEG-PCLA (1700-1500-1700) is 25% w/v in DI water. The optimized PLGA Microspheres (mg):PCLA:PEG:PCLA (mg) ratio is 1:10 w/w.

PCLA-PEG-PCLA (1 g) polymer was dissolved in DI water (4 ml) in room temperature overnight under magnetic stirring (300 rpm). PLGA microspheres were dispersed into PCLA-PEG-PCLA by gentle vortex mixing for 5-10 seconds. The dissolved polymer was incubated at 37° C. for 10 minutes. Gelling was confirmed if there was no flow of the formulation after inverting the tube for 60 seconds

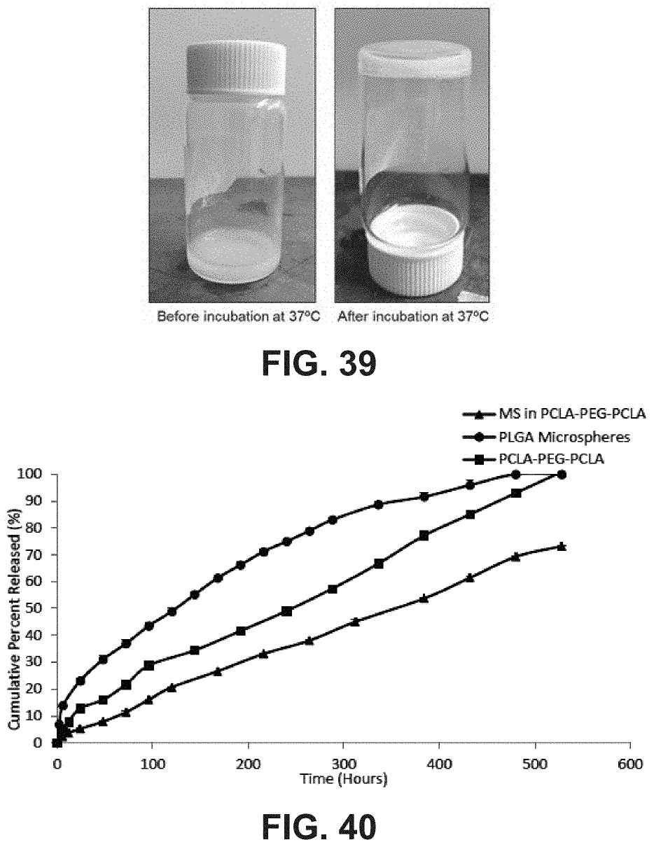

FIG. 39 shows PLGA microspheres dispersed in PCLA-PEG-PCLA Thermogel before and after incubation at 37° C.

In Vitro Release Study

10 mg of PLGA microspheres was dispersed in 400 μl of 25% w/v PCLA-PEG-PCLA. The formulation was incubated at 37 degrees for 10 minutes. 1.3 ml of PBS containing 0.05% w/v SDS was added as release medium. 1 ml of medium was replaced at each time points with fresh buffer to maintain sink conditions. The drug concentration in release medium was analyzed using HPLC.

FIG. 40 shows an in vitro release profile of 4-hydroxy tamoxifen from PLGA microspheres, PCLA-PEG-PCLA Thermogel and Microspheres in PCLA-PEG-PCLA Thermogel formulations. PCLA-PEG-PCLA is poly(ε-caprolactone-co-lactide)-b-poly(ethylene glycol)-b-poly(ε-caprolactone-co-lactide).

For In vivo studies, the levels of 4-hydroxytamoxifen and endoxifen were measured using LC-MS.

Plasma Extraction

For blood concentration, 100 μl of plasma was mixed with 400 μl of acetonitrile to precipitate proteins (1:4 v/v). This was then vortexed for 1-2 minutes followed by sonication for 3 minutes. The plasma was then centrifuged at 10,000 rpm at 4° C. for 10 minutes. The supernatant was collected and evaporated to dryness under gentle stream of Argon. The residue was then reconstituted with 100 μl of Ammonium Formate, pH 3.5: Acetonitrile (7:3 v/v). This was vortexed for 1 minute followed by sonication for 2 minutes. The reconstituted mixture was centrifuged at 10,000 rpm for 10 minutes at 4° C. The supernatant was collected and 15 μl was injected into LC-MS to determine drug levels.

Organ Extraction

The tissues were mixed with TRIS HCl buffer (1 ml/mg) and homogenized at 6000 rpm for 3 minutes under ice. For lymph nodes, sonication (45% amplitude in pulse mode 1 sec on and 1 sec off for 2 minutes under ice) was used to homogenize the tissue. From this, 200 μl of tissue homogenate was mixed with 800 μl of acetonitrile to precipitate proteins (1:4 v/v). This was then vortexed for 1-2 minutes followed by sonication for 3 minutes. The plasma was then centrifuged at 10,000 rpm at 4° C. for 10 minutes. The supernatant was collected and evaporated to dryness under gentle stream of Argon. The residue was then reconstituted with 100 μl of Ammonium Formate, pH 3.5: Acetonitrile (7:3 v/v). This was vortexed for 1 minute followed by sonication for 2 minutes. The reconstituted mixture was centrifuged at 10,000 rpm for 10 minutes at 4° C. The supernatant was collected and 15 μl was injected into LC-MS to determine drug levels.

In Vivo Studies

RESULTS: Mammary glands treated with PLGA microspheres in PCLA-PEG-PCLA showed greater retention at all-time points studied with respect to control. Free 4-hydroxy tamoxifen was found to be retained at very low levels in the mammary gland after 7 days. The levels of 4HT was >3000 fold higher than free 4-hydroxytamoxifen at day 7 and >1000 fold higher at day 28. Endoxifen, a metabolite of 4-hydroxytamoxifen was found in the mammary gland at all-time points tested. The endoxifen levels were 60 fold and 22 fold higher than free 4 hydroxy tamoxifen at days 14 and 28 respectively.

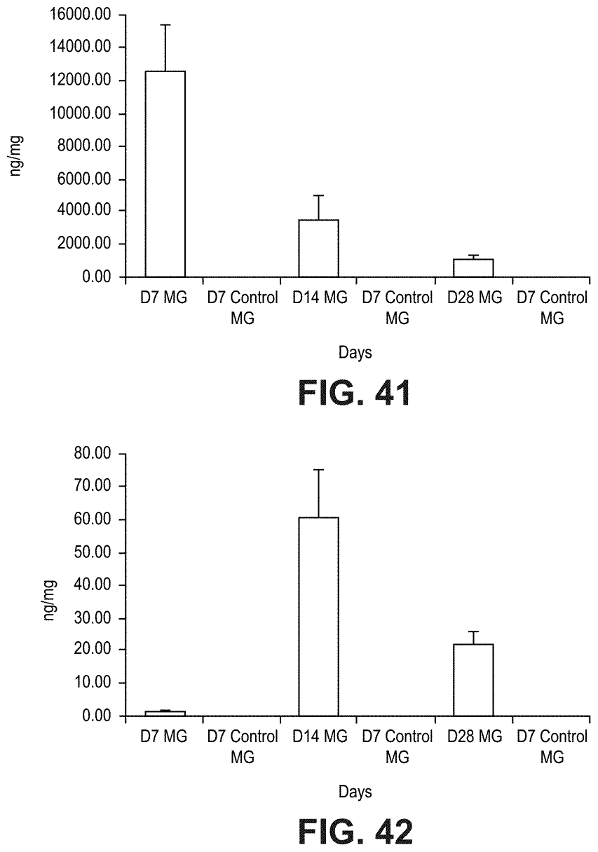

FIG. 41 shows Mammary gland concentration of 4-hydroxytamoxifen treated with PCLA-PEG-PCLA.

FIG. 42 shows Mammary gland concentration of endoxifen treated with PCLA-PEG-PCLA.

FIG. 43 shows Mammary gland concentration of 4-hydroxytamoxifen treated with free 4-hydroxytamoxifen.

Plasma Concentration of 4-Hydroxytamoxifen and Endoxifen

RESULTS: The plasma levels of endoxifen were higher than 4-hydroxytamoxifen in both the treatment groups. In MS in Thermogel, the ratio of endoxifen and 4-hydroxytamoxifen was close to 2 fold higher at day 7 and day 14. At day 7, 4-hydroxytamoxifen and endoxifen was detected in the plasma at lower levels.

FIG. 44 shows Plasma concentration of 4-hydroxytamoxifen treated with PCLA-PEG-PCLA.

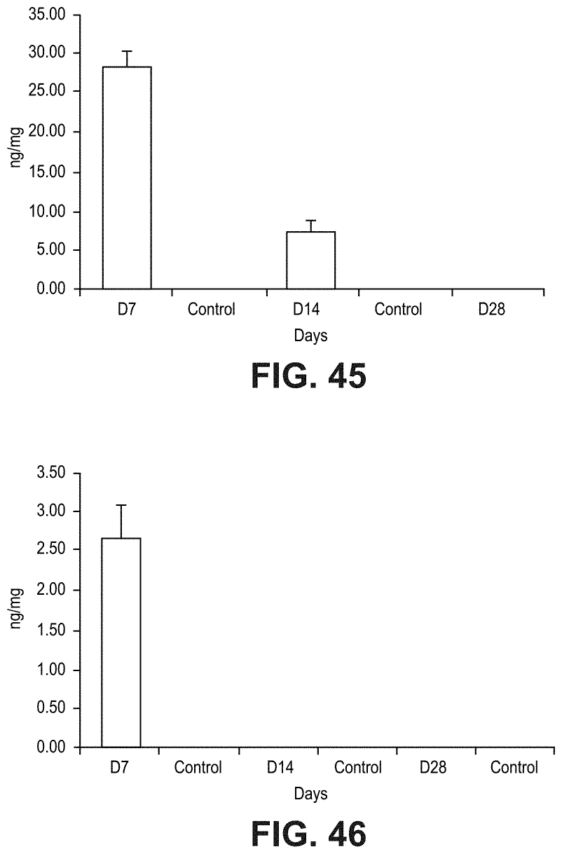

FIG. 45 shows Plasma concentration of endoxifen treated with PCLA-PEG-PCLA.

FIG. 46 shows Plasma concentration of 4-hydroxytamoxifen treated with free 4-hydroxytamoxifen.

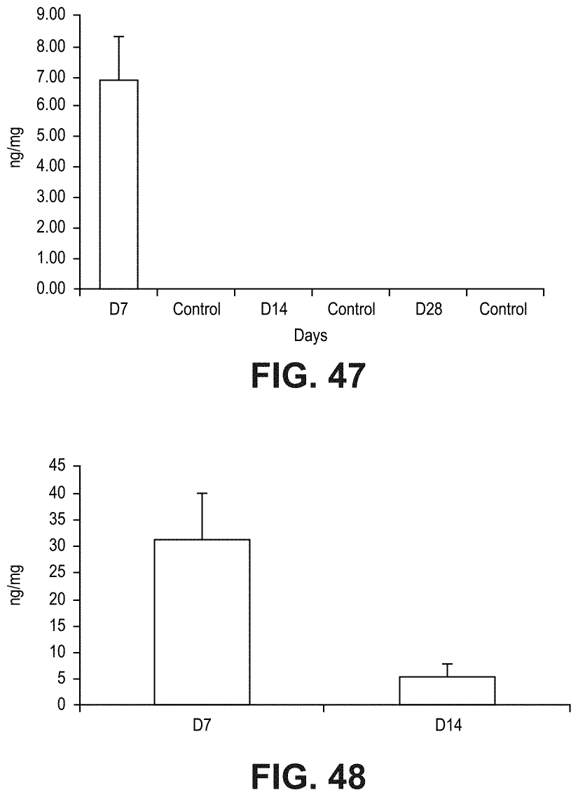

FIG. 47 shows Plasma concentration of endoxifen treated with free 4-hydroxytamoxifen.

Lymph Node Retention Study

RESULTS: The level of 4-hydroxytamoxifen in the thermo gel group was 30 and 5 fold higher in the regional lymph nodes compared to free 4-hydroxytamoxifen. Endoxifen was present at very low levels in the regional lymph nodes at day 7 in mammary glands treated with thermo gel.

FIG. 48 shows Lymph node concentration of 4-hydroxytamoxifen in rats treated with Microspheres in PCLA-PEG-PCLA Thermogel.

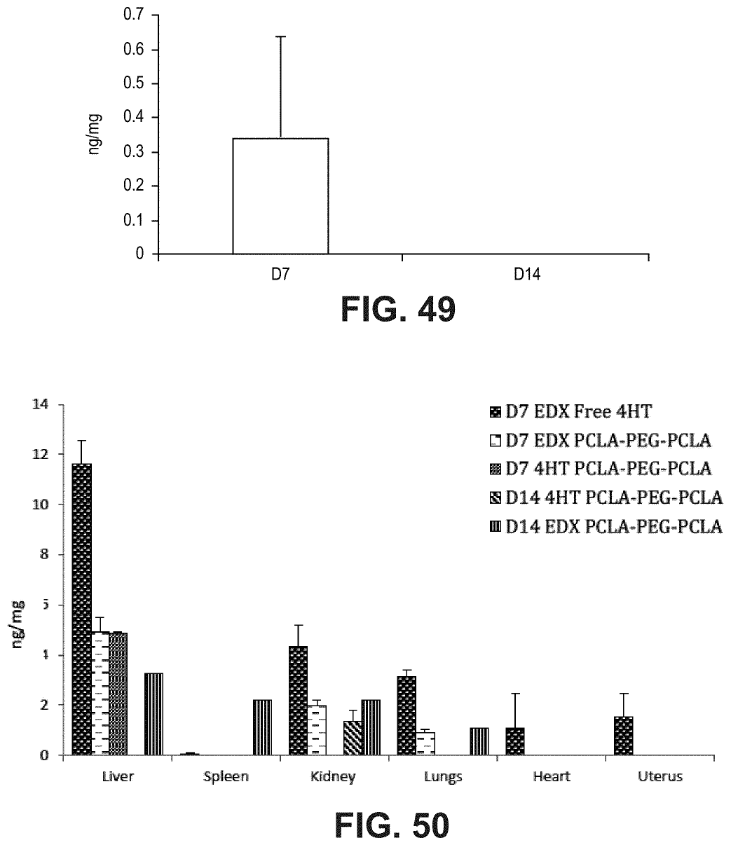

FIG. 49 shows Lymph node concentration of endoxifen in rats treated with Microspheres in PCLA-PEG-PCLA Thermogel.

Biodistribution of Formulations

RESULTS: The concentration of endoxifen was higher in all the treatment groups. Free 4-hydroxy tamoxifen group showed higher systemic exposure of endoxifen in all the tissues and 4 hydroxy tamoxifen was undetectable at day 7. The drug levels were undetectable at day 28.

Thermogel formulation did not result in the uterine exposure of drugs

FIG. 50 shows Organ distribution of 4-hydroxytamoxifen and endoxifen in rats treated with free 4-hydroxy tamoxifen and PCLA-PEG-PCLA at Days 7 and 14.

1. An anticancer composition comprising an anti-cancer agent and poly(lactic-co-glycolic acid) (PLGA) carrier thereof.

2. The composition of claim 1, wherein the carrier is a microsphere.

3. The composition of claim 2, wherein the microsphere is comprised of a polymer of about 75-85 KDa and wherein the particle size of the micro sphere ranges from about 1 to about 50 μm.

4. The composition of claim 1, wherein the PLGA is comprised of lactic acid and glycolic acid present at a ratio of about 75:25.

5. The composition of claim 1, wherein the carrier is a nanoparticle, and wherein the nanoparticle size ranges from about 1 to about 1000 nm.

6. The composition of claim 6, wherein the particle size of the nanoparticle is approximately 200 nm.

7. The composition of claim 1, wherein the composition further comprises a thermogel comprising poly(ε-caprolactone-co-lactide)-b-poly(ethylene glycol)-b-poly(ε-caprolactone-colactide) (PCLA-PEG-PCLA), and wherein the PLGA carrier is in the form of microspheres, nanoparticles, or combinations thereof.

8. The composition of claim 10, wherein the PLGA is present at about 10% w/w.

9. The composition of claim 8, wherein the thermogel is comprised of a polymer with a PCLA:PEG:PCLA molecular weight ratio of 1700:1500:1700 Da, and wherein the thermogel exhibits sustained release of the anti-cancer agent upon injection into a subject.

10. The composition of claim 9, wherein the anti-cancer agent is tamoxifen and wherein the delivery of the composition to the subject produces sustained exposure of the site of delivery to 4-hydroxy tamoxifen and endoxifen.

11. A method for treating a breast disorder in a subject in need thereof, the method comprising administering to the breast of a subject via an intraductal injection an effective amount of a composition comprising a therapeutic agent and a PLGA carrier thereof.

12. The method of claim 11, wherein the composition forms an in situ gel implant upon injection into the subject and wherein the composition is retained in the breast duct and exhibits sustained release of the therapeutic agent therein.

13. The method of claim 11, wherein the breast disorder is breast cancer and the therapeutic agent is an anti-cancer agent.

14. The method of claim 13, wherein the anti-cancer agent is select from a list consisting of: a selective estrogen receptor modulator selected from: tamoxifen, 4-hydroxy tamoxifen, endoxifen, and fulvestrant); a retinoids (e.g. fenretinide); a chemotherapeutic agent selected from fluorouracil, paclitaxel, and cyclophosphamide; and Herceptin, and combinations thereof.

15. The method of claim 11, wherein the breast disorder is an infection.

16. method of claim 11, further comprising administering the composition in conjunction with at least one other treatment or therapy.

17. The method of claim 16, wherein the other treatment or therapy comprises co-administering an anti-cancer agent.

18. The method of claim 16, wherein the other treatment or therapy comprises co-administering α-santalol.

19. A method for treating a lymph node disorder in a subject in need thereof, the method comprising administering to the breast of a subject via an intraductal injection an effective amount of a composition comprising a therapeutic agent and a PLGA carrier thereof and wherein the composition is retained in the lymph node and exhibits sustained release of the therapeutic agent therein.

20. The method of claim 19, wherein the lymph node disorder is selected from a list consisting of: lymphedema, lymphadenopathy, lymphadenitis, lymphomas, and lymphoproliferative disorders.
